(19) United States
(12) Patent Application Publication
(10) Pub. No.: US 2008/0193481 A1
(43) Pub. Date: Aug. 14, 2008

(54) Synthetic Anti-Candida Albicans Oligosaccharide Based Vaccines

(75) Inventors: David R. Bundle, Edmonton (CA); Xiangyang Wu, Edmonton (CA); Tomasz Lipinski, Edmonton (CA); Robert P. Rennie, Sherwood Park (CA)

Correspondence Address: FOLEY & LARDNER LLP, 975 PAGE MILL ROAD, PALO ALTO, CA, 94304, US

(73) Assignee: Governors of the University of Alberta

(21) Appl. No.: 11/908,271

(22) PCT Filed: Mar. 14, 2006
(86) PCT No.: PCT/CA2006/000377

§ 371 Date: Dec. 12, 2007

Related U.S. Application Data
(60) Provisional application No. 60/661,851, filed on Mar. 14, 2005.
(60) Provisional application No. 60/676,101, filed on Apr. 29, 2005.
(60) Provisional application No. 60/686,118, filed on May 31, 2005.

(51) Int. Cl. A61K 31/702; A61K 47/00; A61K 39/00; A61P 37/00

(52) U.S. Cl. 424/204.1; 514/54; 514/776

(57) ABSTRACT

The present invention provides immunogenic oligosaccharide compositions and methods of making and using them. In particular, the compositions comprise native O-linked and S-linked oligosaccharides coupled to a protein carrier via a linker, wherein the resultant conjugate elicits a protectively immunogenic response, particularly in vaccines against pathogenic Candida species and more particularly against Candida albicans. Preferably the pathogenic Candida species are those that possess cell wall oligosaccharide compositions similar to the β-mannan component of Candida albicans cell walls.

### CROSS-REFERENCE TO RELATED PATENT APPLICATIONS

This application claims priority under 35 U.S.C. §119(e) to each of U.S. Provisional Application No. 60/661,851, filed Mar. 14, 2005; 60/676,101, filed Apr. 29, 2005; and 60/686,118, filed May 31, 2005, the contents of which are incorporated by reference.

### TECHNICAL FIELD

The present invention provides immunogenic oligosaccharide compositions and methods of making and using them. In particular, the compositions comprise native O-linked and S-linked oligosaccharides coupled to a protein carrier via a linker, wherein the resultant conjugate elicits a protectively immunogenic response, particularly in vaccines against pathogenic Candida species and more particularly against Candida albicans. Preferably the pathogenic Candida species are those that possess cell wall oligosaccharide compositions similar to the β-mannan component of Candida albicans cell walls.

### BACKGROUND ART

Throughout this application, various publications are referred to by an Arabic number. The bibliographic citations for these references can be found at the end of the specification, immediately preceding the claims. The disclosures of these references are incorporated by reference into this application to more fully describe the state of the art to which this invention pertains.

Candida albicans, the most common etiologic agent in candidiasis, commonly affects immunocompromised patients and those undergoing long-term antibiotic treatment.² The number of cases of systemic candidiasis has become a major medical problem in hospitals.² Treatment of these infections is increasingly difficult due to drug resistance and the toxicity of known antifungal compounds.³ Humoral and cell mediated immunity both appear to play roles in host defenses against C. albicans. While most patients with serious mucosal infections have defects in their cellular immunity,⁴ patients with deep tissue invasion seem to lack antibodies against the (1→2)-β-mannan oligomer found in the yeast cell wall.⁵

The β-mannan component of Candida albicans cell walls is a relatively small component of the much larger β-mannan to which it is attached via a phosphodiester group.^{6,7} Notwithstanding the importance of the larger mannan in defining some Candida serogroups, the small β-mannan appears to hold potential as a protective antigen.

The cell wall phosphomannan antigen has received the greatest attention as it is highly immunogenic.¹ The glycan chain of this complex N-linked glycoprotein is composed of an extended (1→6)-α-D-mannopyranan backbone containing (1→2)-α-D-mannopyranan branches attached to which are shorter (1→2)-β-mannopyranan oligomers.^{7,39,40} In Candida albicans both acid labile and acid stable β-mannans are present and function as protective antigens. Attachment of the acid labile β-mannan occurs via a phosphodiester, but the exact attachment point has yet to be determined. Some of the (1→2)-β-mannan oligomers are linked directly to the α-mannan via a glycosidic bond and not via a phosphodiester. Candida albicans serotype B is defined by the acid labile β-mannan, while strains of serotype A have both acid stable and acid labile β-mannan epitopes.^{39,40}

Monoclonal antibodies that protect mice against the pathogenic yeast, C. albicans^{8,9,10} have been shown to be specific for the cell wall (1→2)-β-mannan antigen.^{6,7} Such antibodies raised against C. albicans cell wall extracts in mice were protective against disseminated candidiasis and vaginal candidiasis.⁸⁻¹¹ Further studies on these protective monoclonal antibodies indicated the active antigen to be a (1→2)-β-mannan polymer that is present as a component of the cell wall phopshomannan,¹² and separately as a phospholipomannan.¹³ In both forms, the (1→2)-β-mannan antigen is relatively small comprising 2-14 residues.¹⁴ The immunochemistry and solution properties of this antigen are of great interest since (1→2)-β-mannan oligomers have potential as the key epitope of conjugate vaccines.¹⁵

The rational synthesis of β-mannosides is a longstanding problem in glycoside synthesis, that until recently, lacked a general solution despite several novel approaches.¹⁶⁻¹⁹ In the construction of large homo-oligomers, the separation of anomeric mixtures posed a major obstacle to efficient assembly by either block or sequential chain extension reactions.

### DETAILED DESCRIPTION

The embodiments provide efficient methods for synthesizing (1→2)-β-mannan. In addition, the embodiments provide a method for coupling the synthesized (1→2)-β-mannan to a carrier protein, or hapten, via a linker, for use as an antigen in a vaccine composition for administration to an animal. The desirability of a Candida vaccine is readily apparent to those in the art.

Embodiments provide a conjugate comprising a conjugate comprising:
a plurality of oligosaccharides comprising a (1→2)-β-D-mannopyranose or a (1→2)-β-D-mannopyranose derivative wherein each monosaccharide unit of said oligosaccharide is linked via an inter-glycosidic atom selected from the group consisting of oxygen and sulfur;
a protein carrier; and
a linking group derived from a linking agent;
wherein the linker group covalently attaches each of said plurality of oligosaccharides to the protein carrier.

Embodiments provide a conjugate comprising:
a plurality of oligosaccharides comprising a (1→2)-β-D-mannopyranose or a (1→2)-β-D-mannopyranose derivative wherein each monosaccharide unit of said oligosaccharide is linked via an inter-glycosidic atom selected from the group consisting of oxygen and sulfur;
a protein carrier; and
a linking group derived from a linking agent;
wherein the linker group covalently attaches each of said plurality of oligosaccharides to the protein carrier;
for use in preventing or ameliorating infection by a Candida species.

### BRIEF DESCRIPTION OF THE DRAWINGS

FIG. 1 is a graph showing rabbit serum titration against BSA and trisaccharide/BSA.

FIG. 2 is a graph showing antibody titration against tetrasaccharide-BSA.

FIG. 3 is a graph showing the relative levels of antibodies, as determined by ELISA assay, in 4 Balb/c mice immunized three times with the trisaccharide-tetanus toxoid conjugate 26 on alum.

FIG. 4 is a graph showing the relative levels of antibodies, as determined by ELISA assay, in 4 Balb/c mice immunized three times with the tetrasaccharide-tetanus toxoid conjugate 27 on alum.

FIG. 5 is a graph showing relative levels of antibodies in rabbits immunized with trisaccharide-BSA conjugate 26. Sera were titrated against β-mannose trisaccharide-BSA conjugate 26 using an ELISA assay. Control sera from animals injected with tetanus toxoid did not show substantial specific anti-mannan activity.

FIG. 6 is a graph showing the relative levels of antibodies in rabbits vaccinated with β-mannose tetrasaccharide-tetanus toxoid conjugate 27. Sera were titrated against β-mannose tetrasaccharide-BSA conjugate 27 using an ELISA assay. Control sera from animals injected with tetanus toxoid did not show substantial specific anti-mannan activity.

FIG. 7 is immunofluorescent staining of C. albicans cells by rabbit antibody specific for the trisaccharide conjugate 26 showing that the rabbit antibody binds to antigen presented on the walls of Candida hyphae and budding cells.

FIG. 8 is a graph showing the white blood cell counts of a rabbit following administration of cyclophosphamide.

FIG. 9 is a graph showing a comparison of viable C. albicans cell in different organs of a rabbit, 8 days after challenge. Values reflect the number of colony forming units per grain of tissue. Solid bars represent median value in the group of 5 rabbits vaccinated with trisaccharide conjugate. Hatched bars refer to the control group of 3 rabbits, vaccinated with tetanus toxoid.

FIG. 10 shows comparison of viable C. albicans cells counts in different organs, 8 days after challenge with live fungi. Given values are the number of cfu (colony forming units) per gram of tissue. “Vaccinated” bars represent average values for rabbits vaccinated with trisaccharide conjugate. “Control” bars refer to a control group, vaccinated with tetanus toxoid.

FIG. 11 is a graph of relative antibodies in rabbits immunized with trisaccharide-BSA conjugate. Here, rabbits were vaccinated twice with tetanus toxoid glycoconjugate absorbed on alum, an adjuvant approved for use in humans. Titers were assayed against trisaccharide-BSA conjugate. Control sera from animals injected with tetanus toxoid did not show specific anti-mannan activity and are not plotted for clarity.

FIG. 12 shows immunofluorescent staining of C. albicans cells using rabbit antiserum raised against trisaccharide tetanus toxoid conjugate. Antibodies bind to antigen presented on the walls of Candida hypae and budding cells.

### MODES FOR CARRYING OUT THE INVENTION

The embodiments provide immunogenic oligosaccharide compositions and methods of making and using them. In particular, the compositions comprise oligosaccharides coupled to a carrier protein via a linker. The resultant conjugate elicits an immune response, that is protective against pathogenic Candida species. Prior to describing the embodiments in further detail, the following terms will first be defined:

### Definitions

As used herein, certain terms may have the following defined meanings. As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “saccharide” or “saccharide unit” or “monosaccharide” refers to a single monosaccharide. Monosaccharides are polyhydroxy aldehydes (aldoses) or ketones (ketoses). The term “sugar” can be used synonymously with the term “saccharide.”

The term “inter-glycosidic atom” refers to the atom joining monosaccharides and in this instance intends the atom X where the glycosidic linkage is defined as C1-X-Cx. C1 one refers to carbon 1 of an aldose, X is the interglycosidic atom and Cx is the carbon atom of the adjacent monosaccharide. In the example below the linkage fragment is shown in bold, X is oxygen and Cx is carbon 2 of a mannopyranose. The glycosidic bond is the bond formed between C1 and X. The glycosidic linkage refers to this bond that joins one monosaccharide to the next.

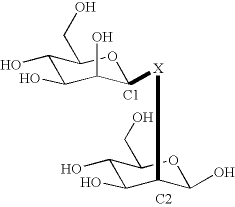

The term “oligosaccharide” refers to a carbohydrate structure having from about 2 to about 14 monosaccharide units wherein each of the monosaccharide units is linked via an inter-glycosidic atom selected from the group consisting of oxygen and sulfur.

The particular monosaccharide units employed are not critical and include, by way of example, all natural and synthetic derivatives of D-mannose, D-glucose, D-galactose, N-D-acetylglucosamine, N-acetyl-D-galactosamine, L-fucose, sialic acid, 3-deoxy-D,L-octulosonic acid, and the like.

In general, a derivative shall intent a compound that can arise from a parent compound by replacement of one atom with another atom or group of atoms. For the purpose of illustration only, derivatives of saccharides include, but are not limited to, saccharides comprising protecting groups. Such derivatives often include a monosaccharide component wherein carbon 1 of the aldose is linked to a protecting group such as an allyl or pentenyl group that can be reacted to provide derivatives that are able to be coupled to protein. Substitution of hydroxyl groups by esterification with long chain carboxylic acids with or without substitution along their alkyl chain for further reactions may be used to enhance immunogencity.

In addition to being in their pyranose form, saccharide units described herein are in their D form except for fucose, which is in its L form.

The term “mannopyranose” refers to a 6-carbon (hexose) sugar having a six-membered ring containing five carbon atoms and one oxygen atom and of formula (I):

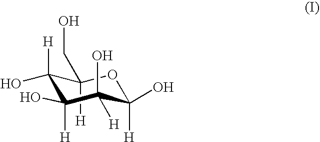

The term “mannopyranose derivatives” refers to mannopyranose as described above with at least one hydrogen of the ring hydroxyl groups is replaced by another chemical moiety. In one embodiment, an acetyl or C2-C6 acyl group or a protecting group such as a benzyl or a p-chlorobenzyl group is used to form a mannopyranose derivative. The acetyl and acyl derivatives can be hydrolyzed in vivo to form a mannopyranose conjugate. The protecting groups, on the other hand, can be removed prior to administration. Mannopyranose derivatives can include mannopyranose with at least one hydrogen of the ring hydroxyl groups replaced by another chemical moiety comprising reactive functional groups.

The term “mannan” with respect to Candida and yeasts in general refer to a complex glycoprotein, a phosphomannan macromolecule that comprises predominantly α-mannopyranose residues N-linked to certain asparagines residues of a protein. Within the carbohydrate moiety there is substantial branching of the backbone α1,6 linked mannopyranose residues by α1,2 linked mannose side chains. The β-mannan component is attached to these side chains. The structure is shown below.

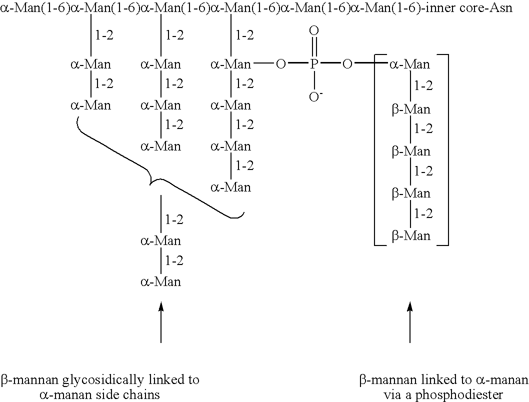

The mannan polysaccharide is predominantly mannose containing and very complex with branching side chains. For the most part the main chain and branches are all-α-linked mannopyranose residues, however in most Candida there is a short chain linked via a phosphate ester to the main α-linked mannan. This short chain varies in length from about 2 β-linked mannopyranose residues to up to about 10-14 β-linked mannopyranose residues. The length depends upon microbial growth conditions.

The term mannan is most often used to mean the whole complex. When part of the complex is being referred to it could be called α-mannan (also called acid stable mannan) or β-mannan. The latter is also called the acid labile mannan because it is easily cleaved off.

The term “protein carrier” or “carrier” refers to a substance that elicits a thymus dependent immune response that can be coupled to a hapten or antigen to form a conjugate. In particular, various protein and/or glycoprotein and/or sub-unit carriers can be used, including, but not limited to, tetanus toxoid/toxin, diphtheria toxoid/toxin, bacteria outer membrane proteins, crystalline bacterial cell surface layers, serum albumin, gamma globulin, and keyhole limpet hemocyanin.

The term “conjugate” refers to oligosaccharides that have been covalently coupled to a protein or other larger molecule with a known biological activity through a linker. The oligosaccharide may be conjugated through the inter-glycosidic oxygen or sulfur.

In the case of the conjugates described, herein, the oligosaccharide is attached through a linker to a protein carrier using chemical techniques providing for linkage of the oligosaccharide to the carrier. In one embodiment, reaction chemistries that result in covalent linkages between the linker and both the protein carrier and the oligosaccharide and are used. Such chemistries can involve the use of complementary functional groups on the hetero- or homo-bifunctional cross-coupling reagent. Preferably, the complementary functional groups are selected relative to the functional groups available on the oligosaccharide or protein carrier for bonding or which can be introduced onto the oligosaccharide or carrier for bonding. Again, such complementary functional groups are well known in the art.

For example, reaction between a carboxylic acid of either the linker or the protein and a primary or secondary amine of the protein or the linker in the presence of suitable, well-known activating agents results in formation of an amide bond; reaction between an amine group of either the linker or the protein and a sulfonyl halide of the protein or the linker results in formation of a sulfonamide bond covalently; and reaction between an alcohol or phenol group of either the linker or the protein carrier and an alkyl or aryl halide of the carrier or the linker results in formation of an ether bond covalently linking the carrier to the linker. Similarly these complimentary reactions can occur between the linker and the oligosaccharide to form a linkage between the oligosaccharide and the linker.

The following Table 1 illustrates numerous complementary reactive groups and the resulting bonds formed by reaction there between.

TABLE 1
Complementary Reactive Groups and Resulting Linkages

| First reactive group | Second reactive group | Resulting linkage |
|---|---|---|
| hydroxyl | isocyanate | urethane |
| amine | epoxide | β-hydroxyamine |
| amine | ketone | Imine |
| amine | ketone | secondary amine |
| sulfonyl halide | amine | Sulfonamide |
| carboxyl | amine | Amide |
| acyl azide | amine | Amide |
| hydroxyl | alkyl/aryl halide | Ether |
| epoxide | alcohol | β-hydroxyether |
| epoxide | sulfhydryl | β-hydroxythioether |
| maleimide | sulfhydryl | Thioether |
| carbonate | amine | Carbamate |
| ketone | aminooxy | oxime |

The term “heterobifunctional cross coupling reagents” refers to a reagent that is used to couple two other molecules or species together by having at least two different functional groups built into one reagent. Such cross coupling reagents are well known in the art and include, for example, X-Q-X′, where each of X and X′ are preferably independently cross coupling groups selected, for example, from —OH, —CO₂H, epoxide, —SH, —N═C═S, and the like. Preferably Q is a group covalently coupling X and X′ having from about 1 to about 20 atoms or alternatively, can be from about 1 to about 15 carbon atoms. Examples of suitable heterobifunctional cross coupling reagents include squarate derivatives, as well as entities derived from succinic anhydride, maleic anhydride, polyoxyalkylenes, adipic acid (CO₂H—C₆—CO₂H), and azelaic acid (CO₂H—C₉—CO₂H). The heterobifunctional cross coupling reagents may also be a lipid or lipid mimic, where the carbohydrate hapten may be covalently linked to the lipid or the lipid is co-administered as an immunological adjuvant.

The term “homobifunctional cross coupling reagents” refers to a reagent that is used to couple two other molecules or species together by having at least two of the same functional groups built into one reagent. Such cross coupling reagents are well known in the art and include, for example, X-Q-X, where X and Q are as defined above. 1,2-diaminoethane, a dicarboxylic acid chloride and diethyl squarate are examples of such homobifunctional cross coupling reagents, as are adipic acid (CO₂H—C₆—CO₂H) and azelaic acid (CO₂H—C₉—CO₂H). Homobifunctional cross coupling reagents may also be derived from lipids and lipid mimics.

The term “linking agent” refers to a reagent that is used to couple two other molecules or species together. Thus, linking agents include heterobifunctional cross coupling reagents and homobifunctional cross coupling reagents. In one embodiment, the linking agent comprises a functional group selected from the “first reactive group” in Table 1. In another embodiment, the linking agent comprises a functional group selected from the “second reactive group” in Table 1. For example, a linking agent can comprise a functional group selected from the “first reactive group” in Table 1 while a mannopyranose derivative can comprise a functional group selected from the “second reactive group” in Table 1, or vice versa.

The term “linker” or “linking group” refers to the residue produced after covalent bonding of the linking agent, homobifunctional cross coupling reagent, or heterobifunctional cross coupling reagent to the oligosaccharide and the protein carrier.

The term “immunogen” refers to a composition used to stimulate an immune response in a mammal. In one aspect, the immunogen confers resistance to the disease or infection in that mammal, which as used herein, infers that the response has immunologic memory. In one aspect, the immunogen is a vaccine.

The term “adjuvant” refers to a non-antigenic substance (including but not limited to aluminum hydroxide, aluminum phosphate, aluminum sulfate, alum, Freund's adjuvant, and RIBI's adjuvant) that, in combination with an antigen, enhances antibody production by inducing an inflammatory response, which leads to a local influx of antibody-forming cells. Adjuvants are used therapeutically in the preparation of vaccines, since they increase the production of antibodies against small quantities of antigen and lengthen the period of antibody production. Some adjuvants are described in U.S. Pat. No. 5,969,130, which is herein incorporated by reference.

The term “immune response” refers to the reaction of the body to foreign or potentially dangerous substances (antigens), particularly disease-producing microorganisms. The response involves the production by specialized white blood cells (lymphocytes) of proteins known as antibodies, which react with the antigens to render them harmless. The antibody-antigen reaction is highly specific. Vaccines also stimulate immune responses.

The term “immunologic memory” refers to the ability of the immune system to remember a previously encountered antigen. Antibodies are produced as a result of the first exposure to an antigen and stored in the event of subsequent exposure.

The term “immunologically effective amount” refers to the quantity of a immune response inducing substance required to induce the necessary immunological memory required for an effective vaccine.

The term “medicament” refers to any suitable pharmaceutical composition. Specifically, it refers to a composition comprising the compound of the embodiments in any suitable excipient or diluent, and also to different formulations for different methods of administration.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

The present oligosaccharide-protein conjugates of the β-mannan component of Candida albicans cell wall antigen are useful in vaccines against any Candida species possessing the β-mannan antigen, particularly C. albicans.

The conjugates of the embodiments may be used as vaccines, as immunogens that elicit specific antibody production or stimulate specific cell mediated immunity responses. They may also be utilized as therapeutic modalities, for example, to stimulate the immune system to recognize tumor-associated antigens; as immunomodulators, for example, to stimulate lymphokine/cytokine production by activating specific cell receptors; as prophylactic agents, for example, to block receptors on cell membrane preventing cell adhesion; as diagnostic agents, for example, to identify specific cells; and as development and/or research tools, for example, to stimulate cells for monoclonal antibody production.

One embodiment provides a conjugate comprising:
a plurality of oligosaccharides comprising a (1→2)-β-D-mannopyranose or a (1→2)-β-D-mannopyranose derivative wherein each monosaccharide unit of said oligosaccharide is linked via an inter-glycosidic atom selected from the group consisting of oxygen and sulfur;
a protein carrier; and
a linking group derived from a linking agent;
wherein the linker group is covalently attached to each of said plurality of oligosaccharides and the protein carrier.

In the embodiments, a protein carrier is a substance that elicits a thymus dependent immune response that can be coupled to a hapten or antigen to form a conjugate. In particular, various protein and/or glycoprotein and/or sub-unit carriers can be used, including, but not limited to, tetanus toxoid/toxin, diphtheria toxoid/toxin, bacteria outer membrane proteins, crystalline bacterial cell surface layers, serum albumin, gamma globulin, and keyhole limpet hemocyanin. Other protein carriers include, but are not limited to, bovine serum albumin, human serum albumin, tetanus toxoid, a recombinant outer membrane class 3 porin (rPorB) from group B Neisseria meningitidis, and T-cell peptide carriers, such as PADRE. In some embodiments, the protein carrier comprises one or more lysine side chains.

In the embodiments, a linking group is the residue produced after covalent bonding of the linking agent, homobifunctional cross coupling reagent, or heterobifunctional cross coupling reagent to the oligosaccharide and the protein carrier. A linking agent is a precursor to the linking group. For example, a heterobifunctional cross coupling reagent or a homobifunctional cross coupling reagent is a linking agent.

In some embodiments, the linking agent has at least three sites of attachment, one of which is reacted to form a linking group for covalent conjugation to the protein carrier. In some embodiments, the linking agent comprises at least two hydroxyl groups. In one embodiment, the linking agent comprises a functional group selected from the “first reactive group” in Table 1. In another embodiment, the linking agent comprises a functional group selected from the “second reactive group” in Table 1. In one embodiment, the linking group is about 1 to about 20 atoms at its longest chain. In some embodiments, the linking agent is a dicarboxylic acid, such as, but not limited to adipic acid and azelaic acid. In some embodiments, linking agent is a p-nitrophenyl adipic acid diester. In some embodiments, the linking agent comprises a sugar having at least one free hydroxyl, such as, but not limited to glucose.

In the embodiments, an oligosaccharide is a carbohydrate structure having from about 2 to about 14 saccharide units wherein each of the saccharide units is linked via an inter-glycosidic atom selected from the group consisting of oxygen and sulfur. The particular saccharide units employed are not critical and include, by way of example, all natural and synthetic derivatives of D-mannose, glucose, galactose, N-acetylglucosamine, N-acetyl-galactosamine, fucose, sialic acid, 3-deoxy-D,L-octulosonic acid, and the like. In some embodiments, the oligosaccharide is selected from the group consisting of disaccharide through hexasaccharide of (1→2)-β-D-mannopyranose and disaccharide through hexasaccharide of(1→2)-β-D-mannopyranose derivatives. In some embodiments, the oligosaccharide is β-D-mannopyranose-(1→2)-β-D-mannopyanose-(1→2)-β-D-mannopyranose. In some embodiments, the oligosaccharide is β-D-mannopyranose-(1→2)-β-D-mannopyranose.

In one embodiment, the conjugate has the structure:

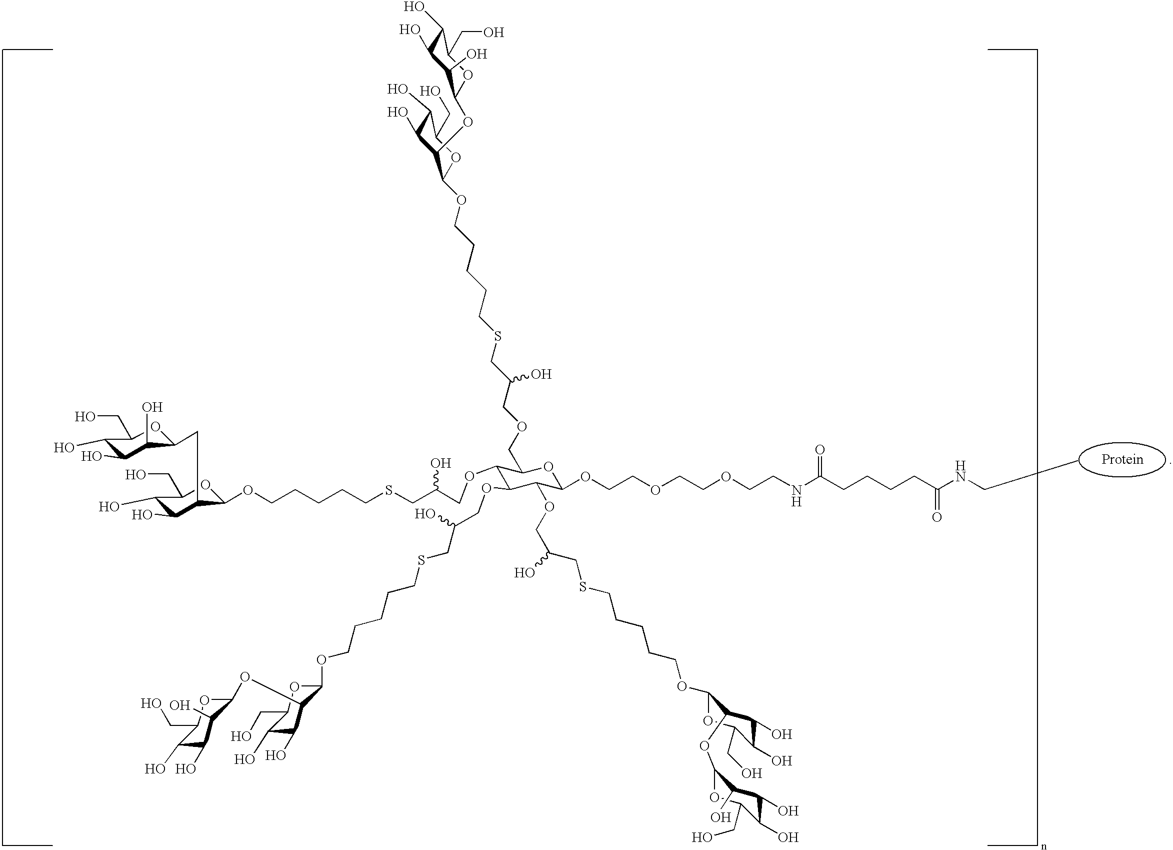

Embodiments provide immunogen comprising a conjugate comprising:
a plurality of oligosaccharides comprising a (1→2)-β-D-mannopyranose or a (1→2)-β-D-mannopyranose derivative wherein each monosaccharide unit of said oligosaccharide is linked via an inter-glycosidic atom selected from the group consisting of oxygen and sulfur;
a protein carrier; and
a linking group derived from a linking agent;
wherein the linker group is covalently attached to each of said plurality of oligosaccharides and the protein carrier; and
a pharmaceutically acceptable carrier.

In some embodiments, the immunogen further comprises a pharmaceutically acceptable adjuvant. In some embodiments, the pharmaceutically acceptable adjuvant is selected from the group consisting of alum, aluminum phosphate, aluminum hydroxide, aluminum sulfate, stearyl tyrosine, Freund's adjuvant, and RIBI's adjuvant.

Embodiments provide a method for inducing an immune response against a Candida species comprising administering to a mammal an immunogenic effective amount of an immunogen comprising a conjugate comprising:
a plurality of oligosaccharides comprising a (1→2)-β-D-mannopyranose or a (1→2)-β-D-mannopyranose derivative wherein each monosaccharide unit of said oligosaccharide is linked via an inter-glycosidic atom selected from the group consisting of oxygen and sulfur;
a protein carrier; and
a linking group derived from a linking agent;
wherein the linker group is covalently attached to each of said plurality of oligosaccharides and the protein carrier; and
a pharmaceutically acceptable carrier.

In one embodiment, the Candida species is Candida albicans.

In one embodiment, the conjugate is administered directly to a urogenital tract.

Embodiments provide a method to induce an immune response comprising administering to a subject in need thereof an immunologically effective amount of the conjugate comprising:
a plurality of oligosaccharides comprising a (1→2)-β-D-mannopyranose or a (1→2)-β-D-mannopyranose derivative wherein each monosaccharide unit of said oligosaccharide is linked via an inter-glycosidic atom selected from the group consisting of oxygen and sulfur;
a protein carrier; and
a linking group derived from a linking agent;
wherein the linker group is covalently attached to each of said plurality of oligosaccharides and the protein carrier; and
a pharmaceutically acceptable carrier.

In one embodiment, the conjugate is administered directly to a urogenital tract.

### Methods and Procedures

Efficient construction of (1→2)-β-mannopyranosides remains a challenging task since despite several novel approaches^{16,18,41} a general solution to the synthesis of this class of molecule has been elusory. Employing 4,6-O-benzylidene-protected mannopyranosyl sulfoxides as a glycosyl donor Crich¹⁹ and coworkers have successfully synthesized a variety of β-mannopyranosyl oligomers including 1,2-linked β-mannopyranosyl oligomers. For our purposes the sulfoxide methodology was not compatible with the allyl protecting group, which was required for transformation into a variety of functionalities late in the synthesis. These reactions provide a tether for coupling to protein, and thus impose limitation on the methods for the synthesis of neoglycoconjugates. Our successful application of ulosyl bromide glycosyl donor and selective reduction developed was especially well suited for the construction of (1→2)-β-mannan oligomers^{20,}²¹ but was most suitable for relatively small-scale synthesis mainly due to the lability of the bromide donor. Gram-scale synthesis of complex β-mannan oligomers for the preparation of neoglycoconjugate is highly demanding. Here, we show that a simplified approach based on the trichloroacetimidate glucosyl donor in combination with an oxidation-reduction strategy is a versatile method for the generation of (1→2)-β-mannan oligomers on a multi-gram scale.

The conjugation strategy whereby oligosaccharide is covalently linked to protein to yield a conjugate vaccine is a major factor that influences the synthetic strategy of oligosaccharide assembly and deprotection.⁴² The chemistry of conjugation may further impart undesirable immunological properties to the vaccine. One of the most efficient coupling methods involves the use of the homobifunctional reagent, diethyl squarate,⁴³ which affords reproducible conjugation in high yields under mild conditions with small amounts of oligosaccharide and protein at low concentration. However, its use in conjugate vaccine application has been correlated with a reduced immune response to the oligosaccharide epitope⁴⁴ and with potential immune response to the squarate residue itself.²³ We have developed a preparation of neoglycoprotein employing the linear homobifunctional p-nitrophenyl ester of adipic acid, which was easily coupled with amino sugar with high efficiency under very mild conditions. Here, trisaccharide and tetrasaccharide glycoconjugate vaccines against Candida albicans were synthesized by this approach.

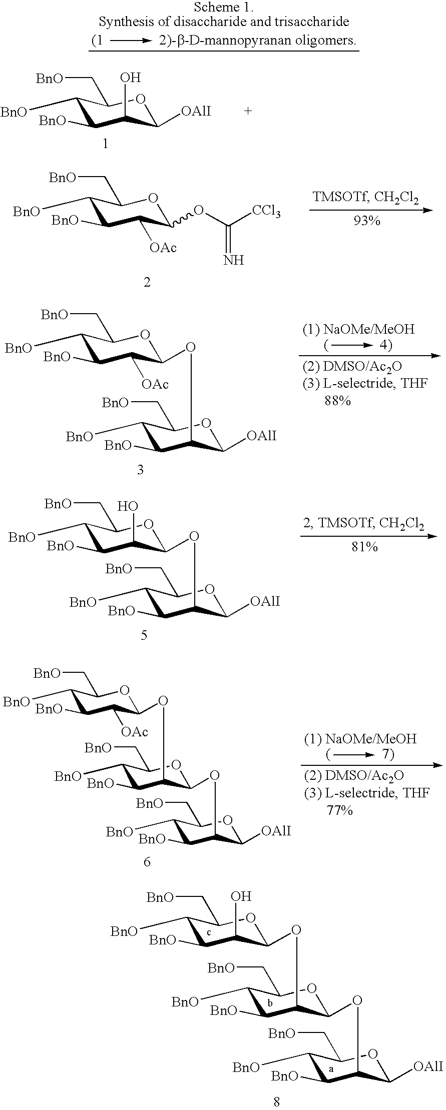

Synthesis of (1→2)-β-mannopyranodisaccharide and trisaccharide.

The synthesis of disaccharide and trisaccharide was accomplished as outlined in Scheme 1. Building blocks 1 (Nitz, M.; Bundle, D. R. J. Org. Chem. 2001, 66, 8411) and 2 (Charette, Andre B.; Turcotte, N.; Cote, B. J. Carbohydr. Chem. 1994, 13, 421; (b) Schmidt, R. R.; Effenberger, G. Liebigs Ann. Chem. 1987, 825) are readily synthesized according to published literature. The glycosylation reaction between monosaccharide acceptor 1 and trichloroacetimidate glycosyl donor 2 was performed by activation with trimethylsilyl trifluoromethanesulfonate (TMSOTf) (about 0.05 equivalents) in CH₂Cl₂ at about −10° C., affording the required disaccharide 3 in excellent yield. Deacetylation under Zemplen conditions gave the desired alcohol 4 quantitively, and this was oxidized by dimethylsulfoxide (DMSO) and acetic anhydride (Ac2O) (2:1). Subsequent selective reduction with L-selectride at about −78° C. in THF afforded the target disaccharide 5 in about 88% yield. After repetition of the glycosylation reaction with donor 2, followed by the saponification, oxidation and reduction sequence, trisaccharide 8 was obtained in about 62% yield over four steps. Excellent diastereoselectivity was observed in this strategy since following reduction only trace amounts of the β-gluco epimer could be detected by ¹H-NMR. This simplified the purification of the product, and most importantly, all the reactions could be performed on a multi-gram scale. Heteronuclear one-bond contants (¹J_{C-H}) were used to unambiguously establish the anomeric configuration of the mannopyransyl residue.

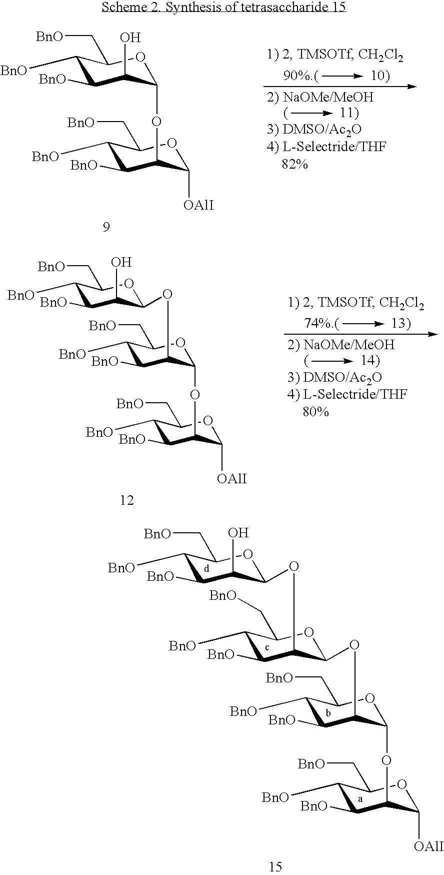

Synthesis of tetrasaccharide man β(1→2)manβ(1→2)manα(1→2)manα(1→2)

In Scheme 2, disaccharide 9 could be conveniently synthesized on a multi-gram scale according to the published procedure (Grathwohl, M.; Schmidt, R. R. Synthesis 2001, 2263). Reaction of disaccharide acceptor 9 with glycosyl donor 2 in CH₂Cl₂ at about −10° C. in the presence of TMSOTf as catalyst (about 0.02 equivalents) afforded the desired trisaccharide 10 in about 90% yield. Trisaccharide 10 was treated under Zemplen deacetylation conditions to give the alcohol 11. Oxidation of the trisaccharide 11 using acetic anhydride and DMSO, followed by reduction with L-selectride at about −78° C. in THF gave trisaccharide 12 in good yield with high selectivity (Scheme 2).

Glycosylation of trisaccharide 12 with donor 2 in the presence of TMSOTf (about 0.02 equivalents) in CH₂Cl₂ at about −10° C. gave the required tetrasaccharide 13 in good yield. Subsequent deacetylation in a mixed solvent of CH₂Cl₂ and MeOH (about 1:1) gave the β-glucopyrannosyl alcohol 14. Final oxidation and reduction as above afforded the desired tetrasaccharide 15 in about 80% yield. The β-(1→2)-mannopyranosyl trisaccharide 8 and tetrasaccharide 15 were obtained on a gram scale.

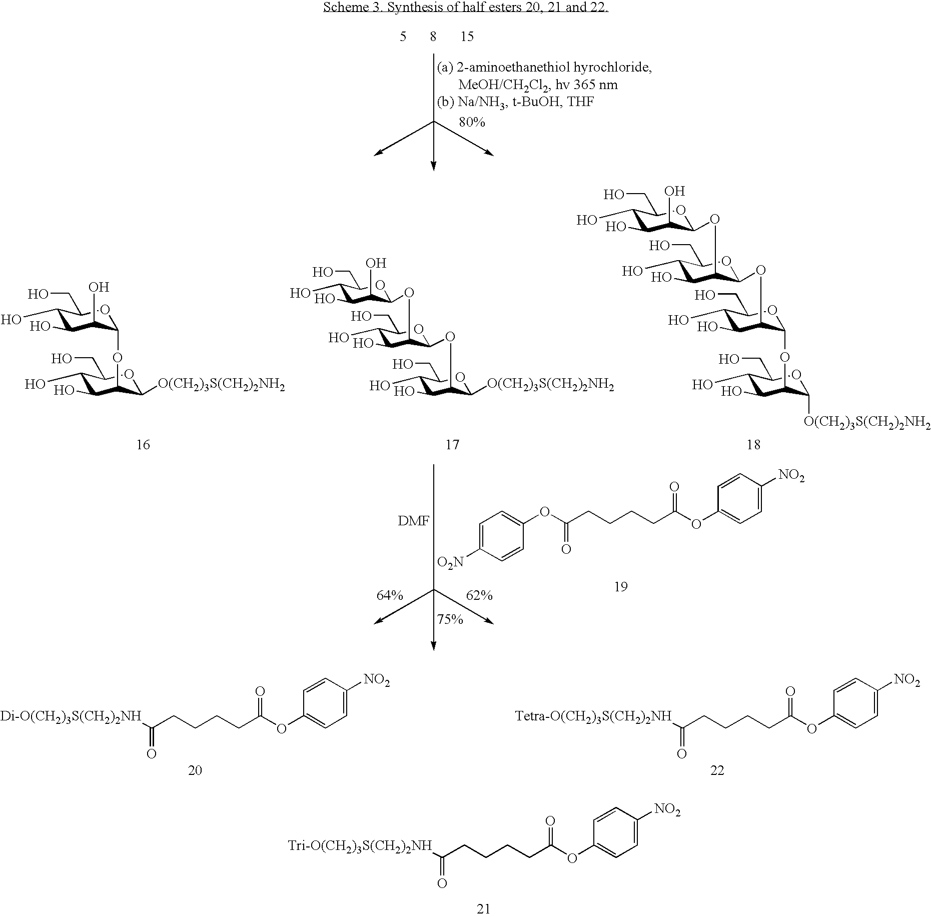

### Synthesis of Half Esters 20, 21 and 22.

For the conjugation of deprotected oligosaccharide to protein, a terminal amine was chosen as a versatile functionality from which glycoconjugates could be readily generated. The protected oligosaccharides 5, 8 and 15 were elaborated via photoaddition of 2-aminoethanethiol to the allyl glycosides to give the amine-functionalized glycosides, then subsequent deprotection under Birch conditions achieved the desired amino-functionalized glycosides 16, 17 and 18 in good yields. Previously, coupling of such compounds to bovine serum albumin (BSA) protein was achieved through a squarate linker. Half esters of adipic acid phenyl ester can be prepared according to recently published procedure (Wu, X.; Ling, C. C.; Bundle, D. R. Org. Lett. 2004, 6, 4407). The oligosaccharide amines 16, 17 and 18 were treated with 5 equivalents of linear homobifunctional p-nitro phenyl ester 19 in dry DMF at about room temperature for about 5 h, affording the corresponding half esters 20, 21 and 22 in good yield after purification on reverse phase column, as shown in Scheme 3. The reaction is readily monitored by TLC or UV spectroscopy, and the half esters are stable to both silica gel chromatograph and reverse-phase isolation under acidic conditions. Excess linker could be removed easily by washing with dichloromethane and the yields of this reaction were in the range of about 62-75%.

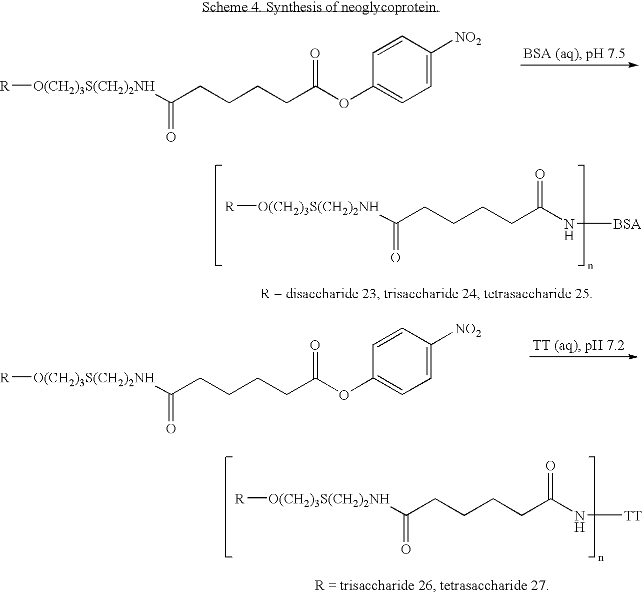

### Formation of Neoglycoproteins.

With the required half esters coupling of 20, 21 and 22 to BSA was performed by an about 18 h incubation in buffer (pH=about 7.5) at ambient temperature. The BSA conjugates 23, 24 and 25 were obtained as white powders after dialysis against deionized water followed by lyophilization (Scheme 4). In the same way, 21 and 22 were conjugated to tetanus toxoid (TT) in phosphate buffer (pH=about 7.2) overnight at ambient temperature. After dialysis against phosphate buffered saline (PBS) pH=about 7.2, the conjugates 26 and 27 were obtained for use as a vaccine. Targeted and observed incorporations are tabulated below (Table 3). The degree of incorporation of the oligosaccharides on BSA or tetanus toxoid was established by MALDI-TOF MS using sinapinic acid as the matrix, and conjugation efficiencies of between about 32.5 and about 44% were achieved, similar to those published for the coupling of oligosaccharides to BSA.²⁵ This corresponds to the incorporation of 12 ligands to TT or BSA with a 30-fold molar excess of activated oligosaccharides.

### Synthesis of a Cluster Conjugate

The synthesis of clustered epitopes utilized a derivative of glucose 34 that was a triethylene glycol glycoside of glucose. The terminal hydroxyl group of the triethylene glycol moiety was derivatized as an azide that served as a latent amino group for the eventual establishment of a covalent linkage to an immunogenic protein carrier. Allylation and subsequent epoxidation yielded racemic 36.

The antigenic epitope was synthesized as a pentenyl glycoside. The method of oligosaccharide assembly followed the procedure described for the synthesis of the corresponding allyl glycosides. On completion of oligosaccharide assembly and following removal of benzyl protecting groups by Birch reduction, which preserved the pentenyl double bond, the oligosaccharide was peractylated. Thioacetic acid was then added across the double bond to yield thioacetate 33.

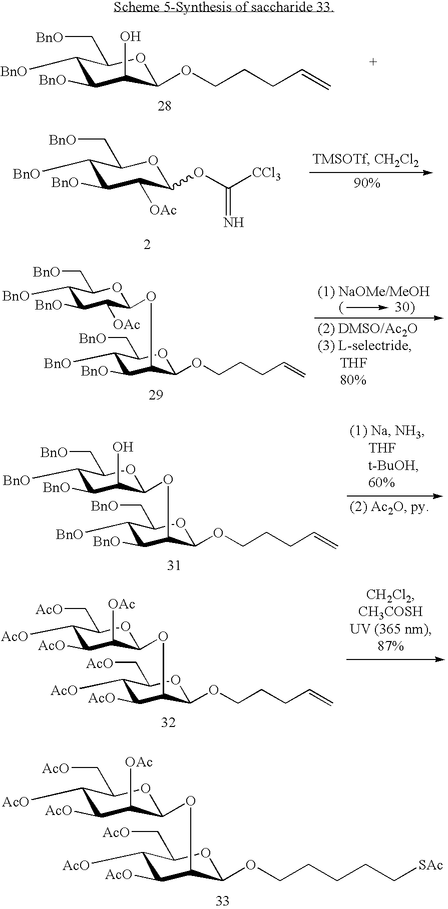

### Synthesis of Saccharide 33.

The glucosyl trichloroacetimidate donor 2 was employed to establish a β-glucopyranosyl linkage to the pentenyl glycoside 28 [Rodebaugh, R.; Debenham, J. S.; Fraser-Reid,; Snyder, J. P.; J. Org. Chem., 1999, 64, 1758-1761] to yield disaccharide 29. Subsequent Swern oxidation and selective reduction facilitated an efficient approach to the β-mannopyranosides 31, which was transformed to compound 32 by Birch reduction and acetylation. Compound 32 was converted then into 33 by UV mediated addition of thioacetate.

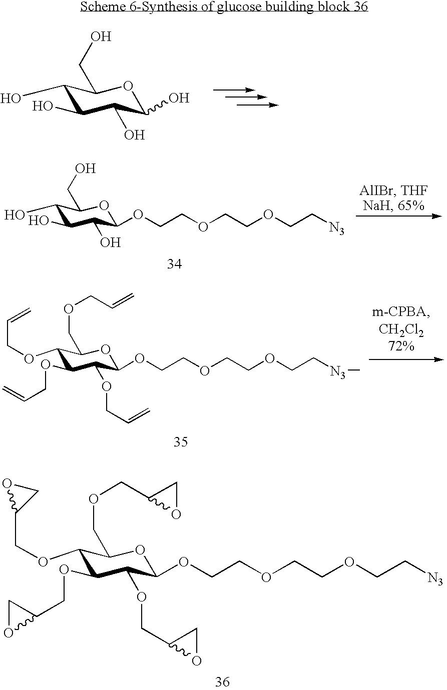

### Synthesis of Glucose Building Block 36

Compound 34 is prepared by a published literature procedure [Kitov, P. I., Tsvetkov, Yu. E, Bakinovsky, L. V. Dokl. Chem. (Engl Transl.) 1993, 329, 1-3]. Reaction of 34 with AllBr in THF with NaH afforded compound 35 in good yield. Subsequent epoxidation with m-CPBA furnished intermediate 36 for coupling with compound 33.

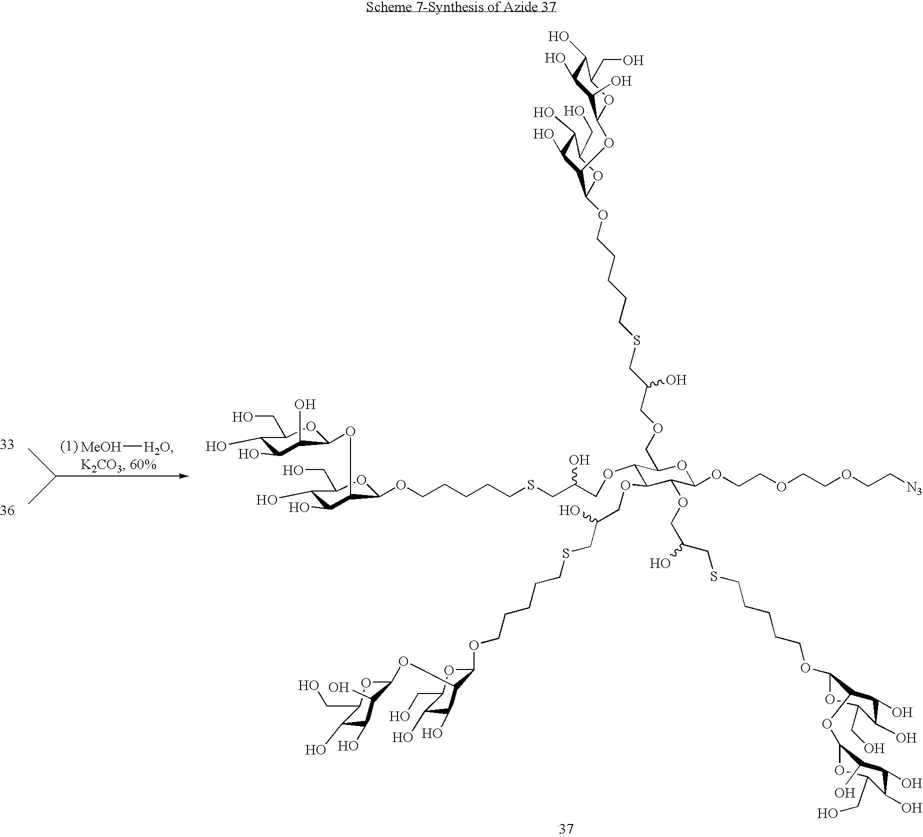

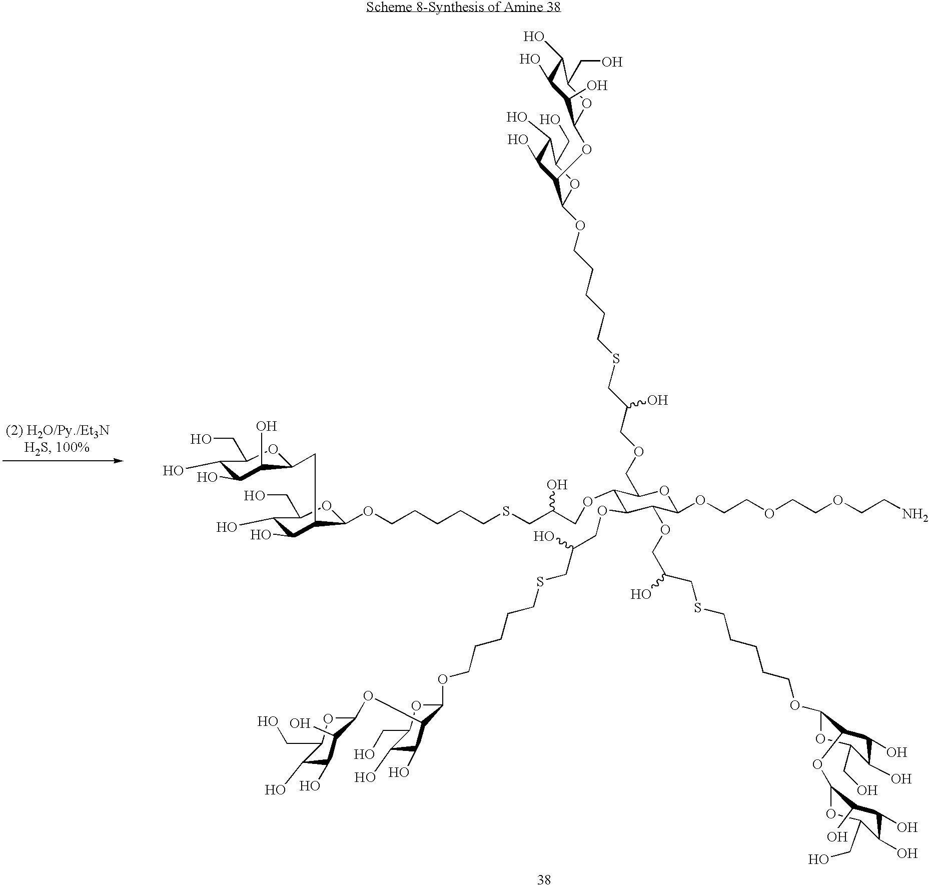

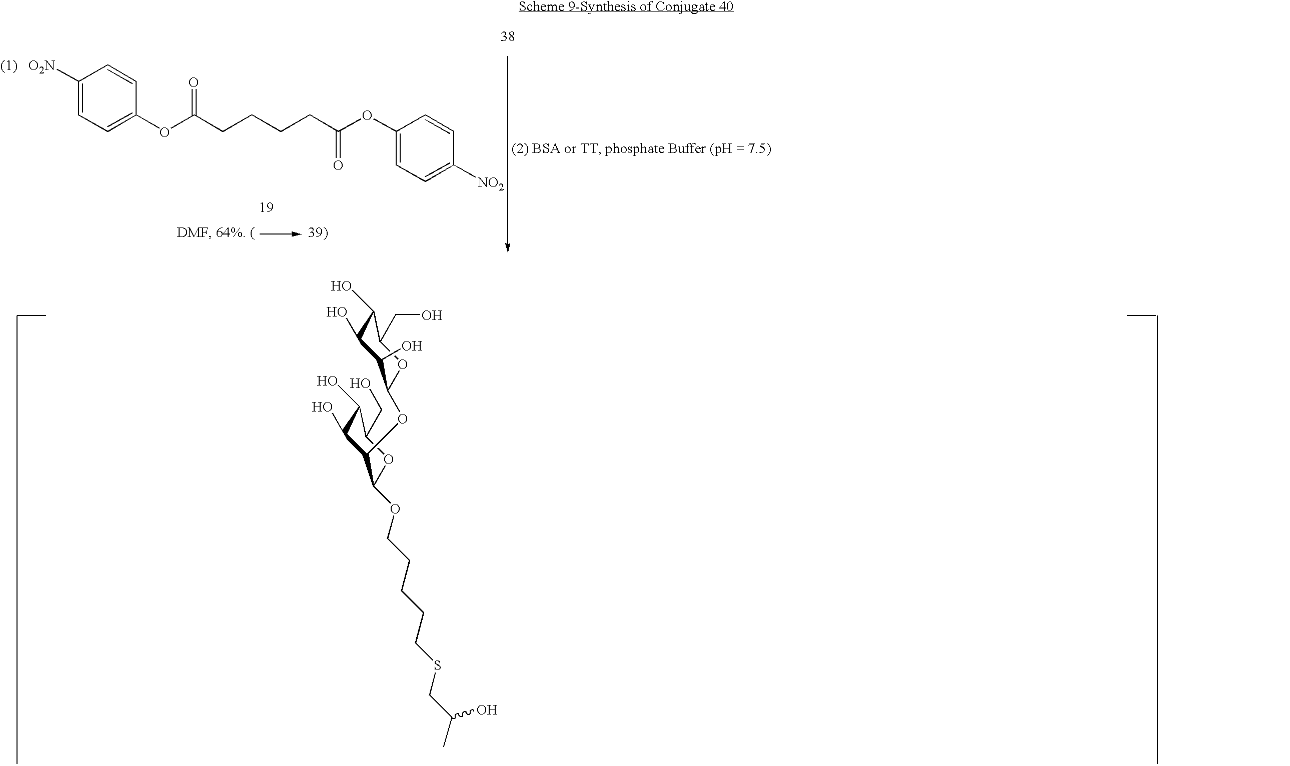

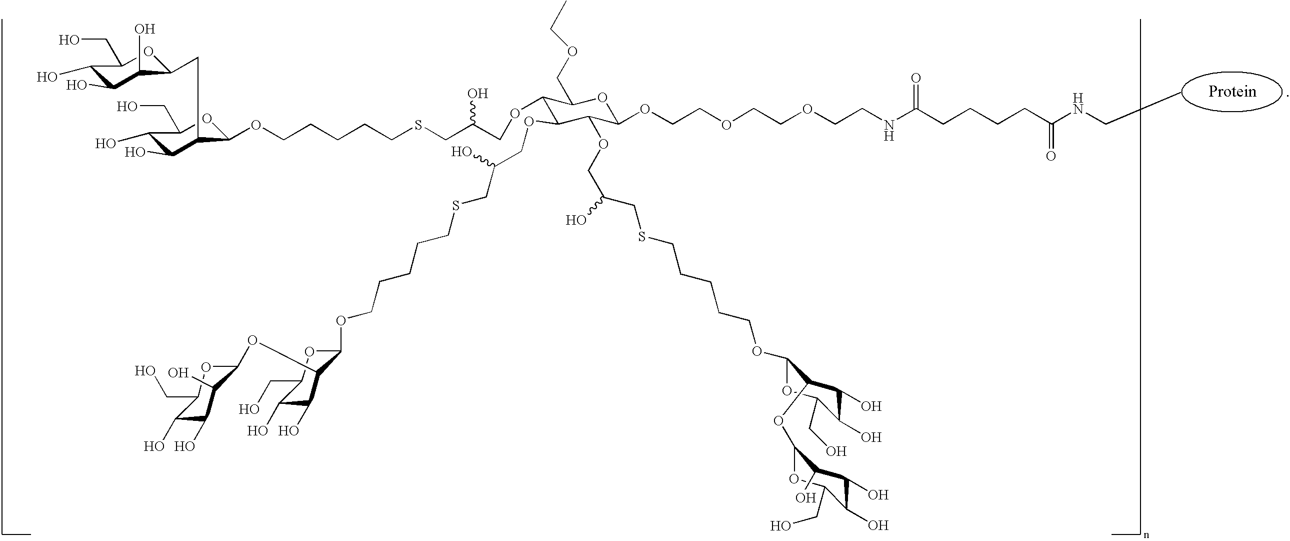

### Conjugation Chemistry

Epoxides 36 and 33 were coupled under basic conditions to give azide 37, as shown in Scheme 7. As shown in Schemes 8 and 9, reduction of the azide provides the primary amine 38 that is sequentially coupled to linker 19 and the desired protein to afford conjugate 40.

The cluster was created by reaction of 33 and 36 under conditions that achieved in situ generation of a thiol, which immediately added to the epoxides of 36. Racemic 38 was obtained after reduction of the terminal azide of 37. This amino-terminated cluster is activated by the p-nitrophenyl adipic acid diester 19 and the activated half ester 39 is purified and coupled directly to either tetanus toxoid or BSA to produce conjugates of 40 for vaccination and/or other uses.

### Pharmaceutical Compositions

The pharmaceutical compositions of the embodiments are advantageously administered in the form of injectable compositions. A typical composition for such purpose comprises a pharmaceutically acceptable carrier. For instance, the composition may contain human serum albumin in a phosphate buffer containing NaCl, e.g., PBS. Other pharmaceutically acceptable carriers include aqueous solutions, non-toxic excipients, including salts, preservatives, buffers and the like (Remington's Pharmaceutical Sciences, Mace Publishing Company, Philadelphia, Pa. 17.sup.th ed. (1985) and The National Formulary XIV, 14^{th} Ed., American Pharmaceutical Association, Washington, DC (1975), both hereby incorporated by reference). Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. The pH and exact concentration of the various components the pharmaceutical composition are adjusted according to routine skills in the art. (Goodman and Gilman, The Pharmacological Basis for Therapeutics, 7th ed., Macmillan Publishing, New York, N.Y., 1985, herein incorporated by reference).

Typically, such vaccines are prepared as injectables, either as liquid solutions or suspensions; solid forms suitable for solution in, or suspension in, liquid prior to injection may also be prepared. The preparation also may be emulsified. The active immunogenic ingredient is often mixed with an excipient that is pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH-buffering agents, adjuvants or immunopotentiators that enhance the effectiveness of the vaccine.

Adjuvants may increase immunoprotective antibody titers or cell mediated immunity response. Such adjuvants could include, but are not limited to, Freunds complete adjuvant, Freunds incomplete adjuvant, aluminium hydroxide, dimethyldioctadecylammonium bromide, Adjuvax (Alpha-Beta Technology), Inject Alum (Pierce), Monophosphoryl Lipid A (Ribi Immunochem Research), MPL+TDM (Ribi hnmunochem Research), Titermax (CytRx), toxins, toxoids, glycoproteins, lipids, glycolipids, bacterial cell walls, subunits (bacterial or viral), carbohydrate moieties (mono-, di-, tri- tetra-, oligo- and polysaccharide) various liposome formulations or saponins. Combinations of various adjuvants may be used with the conjugate to prepare the immunogen formulation.

In another embodiment, the conjugate is formulated for topical applications including gels, lotions, creams and the like. Topical formulations are well known in the art.

An example of a topical formulation may be prepared as follows in Table 2:

TABLE 2

| Ingredient | Quantity (approximate) | |
|---|---|---|
| Active ingredient | 1-10 | g |
| Emulsifying Wax | 30 | g |
| Liquid Paraffin | 20 | g |
| White Soft Paraffin | 20 to 100 | g |

The white soft paraffin is heated until molten. The liquid paraffin and emulsifying wax are incorporated and stirred until dissolved. The active ingredient is added and stirring is continued until dispersed. The mixture is then cooled until solid.

The vaccines are conventionally administered intraperitoneally, intramuscularly, intradermally, subcutaneously, orally, nasally, parenterally or administered directly to the urogenital tract, preferably topically, to stimulate mucosal immunity. Additional formulations are suitable for other modes of administration and include oral formulations. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders and contain about 10%-95% of active ingredient, preferably about 25-70%.

The term “nit dose” refers to physically discrete units suitable for use in humans, each unit containing a predetermined quantity of active material calculated to produce the desired therapeutic effect in association with the required diluent, i.e., carrier or vehicle, and a particular treatment regimen. The quantity to be administered, both according to number of treatments and amount, depends on the subject to be treated, capacity of the subject's immune system to synthesize antibodies, and degree of protection desired. The precise amounts of active ingredient required to be administered depend on the judgment of the practitioner and are peculiar to each individual. However, suitable dosage ranges are on the order of one to several hundred micrograms of active ingredient per individual. Suitable regimes for initial administration and booster shots also vary but are typified by an initial administration followed in one or two week intervals by one or more subsequent injections or other administration. Annual boosters may be used for continued protection.

### EXAMPLES

The following abbreviations are to be given the following meanings. Abbreviations not defined above or below are to given their standard meaning in the art.

BSA=Bovine serum albumin

° C.=degrees Celcius

cfu=colony-forming units

ELISA=Enzyme-Linked Immunosorbent Assay

g=Grams

H=hour

H+L=heavy and light chains

HPLC=High performance liquid chromatography

IR 120 (H±form)=Strong cation exchange resin in the protonated form

iv=intravenous

mg=Milligram

min=minutes

ml=Milliliters

Nm=nanometers

NMR=Nuclear magnetic resonance

° C.=degrees Celcius

M=molar

Nm=nanometers

PBS=phosphate-buffered saline

PBST=PBS+0.05% Tween 20

PRN=Positive pressure adapter

THF=tetrahydrofuran

TLC=Thin layer chromatography

TMB=3,3′,5,5′-tetramethylbenzidine

TT=tetanus toxoid

UV=ultraviolet

Analytical thin-layer chromatography (TLC) was performed on silica gel 60-F254 (Merck). TLC detection was achieved by charring with 5% sulfuric acid in ethanol. All commercial reagents were used as supplied. Column chromatography used silica gel (SiliCycle, 230-400 mesh, 60 Å), and solvents were distilled. High-performance liquid chromatography (HPLC) was performed using a Waters HPLC system that consisted of a Waters 600S controller, 626 pump, and 486 tunable absorbance detector. HPLC separations were performed on a Beckmann C18 semi-preparative reversed-phase column with a combination of methanol and water as eluents. Photoadditions were carried out using a spectroline model ENF-260C UV lamp and cylindrical quartz vessels. ¹H NMR spectra were recorded at either 400, 500, or 600 MHz, and are referenced to the residual protonated solvent peaks; δ_{H} 7.24 ppm for solutions in CDCl₃, and 0.1% external acetone (δ_{H} 2.225) for solutions in D₂O. Optical rotations were measured with a Perkin-Elmer 241 polarimeter at about 22° C. Mass spectrometric analysis was performed by positive-mode electrospray ionization on a Micromass ZabSpec Hybrid Sector-TOF mass spectrometer. MALDI mass spectrometric analysis of protein glycoconjugates was performed on a Voyager-Elite system from Applied Biosystems.

### Example 1

### Allyl (3,4,6-tri-O-benzyl-2-O-acetyl-β-D-glucopyranosyl)-(1→2)-3,4,6-tri-O-benzyl-β-D-mannopyranoside (3)

Glycosyl donor 2 (1.52 g, 2.4 mmol), monosaccharide acceptor 1 (980 mg, 2 mmol) and activated 4 Å molecular sieves (200 mg) were dried together under vacuum for one hour in a pear-shaped flask (50 mL). The contents of the flask were then dissolved in dichloromethane (10 ml). The suspension was stirred for 10 min. at room temperature under argon, and then the temperature was reduced with a −10° C. bath, and trimethylsilyl trifluorometanesulfonate (18 μl) was added dropwise. After 30 min., the reaction mixture was neutralized with triethylamine and concentrated in vacuum. The residue was purified by flash chromatography (n-hexane/ethyl acetate, 6:1) to afford 3 (1.79 g, 93%) as a white foam; [α]_{D}−31.1° (c 1.0, CHC₃); ¹H NMR (400 MHz, CDCl₃), δ=7.18-7.42 (m,30 H, Ar), 5.89 (m, 1H, OCH₂CH═CH₂), 5.32-5.37 (m, 1H, OCH₂CH═CH₂), 5.20-5.23 (m, 1H, OCH₂CH═CH₂), 5.13 (dd, ³J=8.0 Hz, 9.6 Hz, 1 H, 2b-H), 4.76-4.89 (m, 8 H, 1b-H, 7/2 CH₂Ph), 4.44-4.38 (m, 1 H, OCH₂CH═CH₂), 4.28 (d, J_{1,2}=2.8 Hz, 1 H, 1a-H), 4.0-4.1 (m, 1 H, OCH₂CH═CH₂), 3.76-3.82 (m, 3 H, 3b-H, 5a-H, 6b-H), 3.58-3.73 (m, 6 H, 3a-H, 4a-H, 4b-H, 6′b-H, 6a-H, 6′a-H), 3.52 (dd, ³J=2.8 Hz, 9.2 Hz, 1 H, 2a-H), 3.46 (m, 1 H, 5b-H), 1.98 (s, 3 H, Ac); EMS Calcd. (M+Na) 987.4 found 987.4.

### Example 2

### Allyl (3,4,6-tri-O-benzyl-β-D-glucopyranosyl)-(1→2)-3,4,6-tri-O-benzyl-βD-mannopyranoside (4)

To a solution of 3 (2.55 g, 2.65 mmol) in methanol (20 mL) was added sodium methoxide (14 mg, 0.264 mmol), and stirred overnight at room temperature. The resulting mixture was neutralized with IR 120 (H±form), and concentrated in vacuum. The residue was purified by flash chromatography (n-hexane/ethyl acetate, 4:1) to afford 4 (2.44 g, 100%) as a white foam; [α]_{D}−39.8° (c 1.0, CHCl₃); ¹H NMR (600 MHz, CDCl₃), δ=7.22-7.44 (m, 30 H, Ar), 5.95 (m, 1H, OCH₂CH═CH₂), 5.32-5.37 (m, 1H, OCH₂CH═CH₂), 5.24-5.26 (m, 1H, OCH₂CH═CH₂), 5.09-5.11 (d, ²J=11.4 Hz, 1 H, 1/2 CH₂Ph), 4.83-4.89 (m, 4 H, 2 CH₂Ph), 4.75 (d, J_{1,2}=7.8 Hz, 1 H, 1b-H), 4.66 (d, ²J=12.0 Hz, 1H, 1/2 CH₂Ph), 4.52-4.59 (m, 6 H, 3 CH₂Ph), 4.46-4.48 (m, 2 H, 1a-H, OCH₂CH═CH₂), 4.31 (d, ³J=3.1 Hz, 1 H, 2a-H), 4.08-4.11 (m, 1 H, OCH₂CH═CH₂), 3.94 (t ³J=9.5 Hz, 9.9 Hz, 1 H, 4a-H), 3.66-3.81 (m, 6 H, 2b-H, 3b-H, 6b-H, 6′b-H, 6a-H, 6′a-B), 3.54-3.62 (m, 3 H, 3a-H, 4 b-H, 5b-H), 3.44 (m, 1 H, 5a-H); ¹³C-NMR (125 MHz, CDCl₃), 138.0-138.5, 133.5, 127.4-128.3, 117.7, 104.0 (¹J_{C-H}=162 Hz, C-1b), 99.3 (¹J_{C-H}=156 Hz, C-1a), 85.1, 80.3, 75.7, 75.4, 75.3, 75.0, 74.7, 74.5, 74.3, 73.4, 70.3, 69.9, 69.2; EMS Calcd. (M+Na) 945.3 found 945.4.

### Example 3

### Allyl (3,4,6-tri-O-benzyl-β-D-mannopyranosyl)-(1→2)-3,4,6-tri-O-benzyl-β-D-mannopyranoside (5)

Disaccharide 4 (1.5 g, 1.62 mmol) was dissolved in freshly distilled dimethyl sulfoxide (10 mL) and acetic anhydride (5 mL) was added. The resulting solution was stirred for 18 h at room temperature, and diluted with ethyl acetate, then washed with water, sodium bicarbonate solution and a brine solution. Finally, the solution was concentrated at low pressure to give a yellow syrup. This syrup was dissolved in THF (20 ml) and then cooled to −78° C. under argon. L-selectride (1 M THF, 6 mL) was added dropwise and the reaction was stirred for 15 min. The dry ice bath was removed and the reaction was allowed to warm to room temperature. The reaction mixture was quenched after 15 min. with methanol (2 mL), and diluted with dichloromethane. Washing with a solution of hydrogen peroxide (5%) and sodium hydroxide (1 M) followed by sodium thiosulfate (5%) and sodium chloride solutions gave a clear colorless organic solution. The resulting solution was dried over magnesium sulfate and concentrated to a colorless oil. The residue was purified by flash chromatography (n-hexane/ethyl acetate, 3:1) to afford 5 (1.32 g, 88%) as a white oil. The analytic data of compound 5 were identical with the published values.^{18b}

### Example 4

### Allyl (3,4,6-tri-O-benzyl-2-O-acetyl-β-D-glucopyranosyl)-(1→2)-(3,4,6-tri-O-benzyl-β-D-mannopyranosyl)-(1→2)-3,4,6-tri-O-benzyl-β-D-mannopyranoside (6)

The procedure used was analogous to the preparation of 3, and used glycosyl donor 2 (1.08 g, 1.68 mmol), disaccharide 5 (1.29 g 1.41 mmol), dichloromethane (10 mL), trimethylsilyl trifluoromethanesulfonate (13 μl) and activated 4 Å molecular sieves (200 mg). Column chromatography in n-hexane/ethyl acetate (4:1) gave the trisaccharide 6 (1.59 g, 81%). [α]_{D}−50.2° (c 1.0, CHCl₃); ¹H NMR (600 MHz, CDCl₃), δ=7.03-7.44 (m, 45 H, Ar), 5.86 (m, 1H, OCH₂CH═CH₂), 5.27 (d, J_{1,2}=8.4 Hz, 1 H, 1c-H), 5.17-5.20 (m, 2 H, 2c-H, OCH₂CH═CH₂), 5.08-5.11 (m, 1 H, OCH₂CH═CH₂), 4.94-4.98 (m, 3 H, 3/2 CH₂Ph), 4.80-4.86 (m, 2 H, CH₂Ph), 4.69-4.74 (m, 4 H, 1b-H, 3/2 CH₂Ph), 4.63 (d, ²J=13.2 Hz, 1 H, 1/2 CH₂Ph), 4.48-4.57 (m, 9 H, 2b-H, 4 CH₂Ph), 4.43 (d, ²J=12.0 Hz, 1 H, 1/2 CH₂Ph), 4.40 (s, 1 H, 1a-H), 4.35-4.38 (m, 1 H, OCH₂CH═CH₂), 4.20 (d, ³J=3.0 Hz, 1 H, 2a-H), 3.92 (t, ³J=8.4 Hz, 1 H, 3c-H), 3.65-3.81 (m, 8 H, 4c-H, 5b-H, 5c-H, 6c-H, 6a-H, 6′a-H, 6b-H, 6′b-H), 3.62 (t, ³J=9.6 Hz, 1 H, 4b-H), 3.47-3.55 (m, 3 H, 3a-H, 3b-H, 6′c-H), 3.37 (m, 1 H, 5a-H); ¹³C-NMR (125 MHz, CDCl₃), 138.6-138.2, 133.9, 128.4-127.4, 117.1, 102.4, 101.1, 100.2, 83.6, 80.6, 80.2, 78.3, 75.6, 75.5, 75.3, 75.2, 74.9, 74.7, 74.6, 74.5, 73.4, 73.3, 73.0, 72.0, 70.0, 69.8, 69.6; EMS Calcd. For C₈₆H₉₂O₁₇Na 1419.6 Found 1420.0.

### Example 5

### Allyl (3,4,6-tri-O-benzyl-β-D-glucopyranosyl)-(1→2)-(3,4,6-tri-O-benzyl-β-D-mannopyranosyl)-(1→2)-3,4,6-tri-O-benzyl-β-D-mannopyranoside (7)

The procedure used was analogous to the preparation of 4, and used trisaccharide 6 (1.59 g, 1.14 mmol), sodium methoxide (12 mg), dichloromethane (5 mL), methanol (10 mL). Column chromatography in n-hexane/ethyl acetate (4:1) gave the trisaccharide 7 (1.54 g, 100%). [a]_{D}−54.5 (c 1.0, CHCl₃); ¹H NMR (600 MHz, CDCl₃), δ=7.04-7.46 (m, 45 H, Ar), 5.79-5.94 (m, 1H, OCH₂CH═CH₂), 5.23-5.26 (m, 1 H, OCH₂CH═CH₂), 5.18-5.20 (m, 1 H, OCH₂CH═CH₂), 5.02-5.07 (m, 5 H, 5/2 CH₂Ph), 4.98 (s, 1 H, 1b-H), 4.89 (d, ²J=11.4 Hz, 1 H, 1/2 CH₂Ph), 4.81 (d, J_{1,2}=7.8 Hz, 1 H, 1c-H), 4.63-4.72 (m, 3 H, 3/2 CH₂Ph), 4.39-4.56 (m, 13 H, 9/2 CH₂Ph, 2a-H, 2b-H, 1a-H, OCH₂CH═CH2), 4.15 (t, ³J=9.6 Hz, 1 H, 4b-H), 4.06 (m, 1 H, OCH₂CH═CH₂), 3.87 (t, ³J=9.6 Hz, 1 H, 4a-H), 3.66-3.84 (m, 8 H, 2c-H, 3c-H, 6a-H, 6′a-H, 6b-H, 6′b-H, 6c-H, 6′c-H), 3.52-3.63 (m, 5 H, 5c-H, 3a-H, 4c-H, 3b-H, 5a-H), 3.43 (m, 1 H, 5b-H); ¹³C-NMR (125 MHz, CDCl₃), 139.2-138.1, 133.8, 128.4-127.2, 117.4, 105.3, 100.1, 99.9, 86.7, 80.2, 80.1, 77.3, 75.6, 75.4, 74.9, 74.8, 74.7, 74.6, 74.1, 73.6, 73.4, 73.3, 71.1, 70.3, 70.2, 69.8; EMS Calcd. for C₈₄H₉₀O₁₆Na 1377.6 found 1378.0.

### Example 6

### Allyl (3,4,6-tri-O-benzyl-β-D-mannopyranosyl)-(1→2)-(3,4,6-tri-O-benzyl-β-D-mannopyranosyl)-(1→2)-3,4,6-tri-O-benzyl-β-D-mannopyranoside (8)

The procedure used was analogous to the preparation of 5, and used trisaccharide 7 (1.07 g, 0.79 mmol), dimethyl sulfoxide (10 mL), acetic anhydride (5 mL), THF (10 ml), L-selectride (1 M, 3 mL). Column chromatography in n-hexane/ethyl acetate (5:2) gave the trisaccharide 8 (823 mg, 77%). The analytical data of compound 8 were identical with the published values.^{18b}

### Example 7

### Allyl (2-O-acetyl-3,4,6-tri-O-benzyl-β-D-glucopyranosyl)-(1→2)-(3,4,6-tri-O-benzyl-α-D-mannopyranosyl)-(1→2)-3,4,6-tri-O-benzyl-α-D-mannopyranoside (10)

The procedure used was analogous to the preparation of 3, and used glycosyl donor 2 (1.52 g, 2.37 mmol), disaccharide 9 (1.83 g, 1.98 mmol), dichloromethane (10 mL), trimethylsilyl trifluorometanesulfonate (7 μL), activated 4 Å molecular sieves (200 mg). Column chromatography in n-hexane/ethyl acetate (6:1) gave the trisaccharide 10 (2.49 g, 90%). [a]_{D}+9.6° (c 1.0, CHCl₃); ¹H NMR (600 MHz, CDCl₃), δ=7.19-7.40 (m, 45 H, Ar), 5.79-5.85 (m, 1H, OCH₂CH═CH₂), 5.19-5.23 (m, 1 H, OCH₂CH═CH₂), 5.11-5.13 (m, 1 H, OCH₂CH═CH₂), 5.08 (dd, ³J=8.4 Hz, 9.6 Hz, 1 H, 2c-H), 5.01 (d, J_{1,2}=2.4 Hz, 1 H, 1a-H), 4.97 (d, J_{1,2}=1.8 Hz, 1 H, 1b-H), 4.91 (d, ²J=10.8 Hz, 1 H, 1/2 CH₂Ph), 4.66-4.87 (m, 8 H, 4 CH₂Ph), 4.47-4.62 (m, 8 H, 4 CH₂Ph), 4.42 (d, ²J=10.8 Hz, 1 H, 1/2 CH₂Ph), 4.28 (m, 1 H, 1c-H), 4.19 (t, ³J=5.4 Hz, 1 H, 2a-H), 4.12 (m, 1H, 2b-H), 4.08 (m, 1 H, OCH₂CH═CH₂), 3.98 (dd, ³J=3.0 Hz, 9.0 Hz, 1 H, 3b-H), 3.91-3.93 (m, 2 H, 3a-H, 6a-H), 3.77-3.85 (m, 4 H, 4b-H, OCH₂CH═CH₂, 6′a-H, 6b-H), 3.71-3.74 (m, 3 H, 4a-H, 5b-H, 6′c-H), 3.58-3.68 (m, 4 H, 6c-H, 6′b-H, 4c-H, 5a-H), 3.52 (t, ³J=9.0 Hz, 1 H, 3c-H), 3.88 (m, 1 H, 5c-H), 1.98 (s, 3 H, Ac); ¹³C-NMR (125 MHz, CDCl₃), 99.8, 99.4, 98.2, 82.6, 80.2, 77.9, 77.6, 75.1, 75.0, 74.8, 74.7, 74.4, 73.5, 73.4, 73.0, 72.8, 72.7, 71.9, 71.1, 70.3, 69.4, 67.9; ES HRMS calcd. for C₈₆H₉₂O₁₇Na 1419.623222, found 1419.623151.

### Example 8

### Allyl (3,4,6-tri-O-benzyl-βD-glucopyranosyl)-(1→2)-(3,4,6-tri-O-benzyl-α-D-mannopyranosyl)-(1→2)-3,4,6-tri-O-benzyl-α-D-mannopyranoside (11)

Allyl trisaccharide (2.49 g) 10 was deacetylated using the protocol described above to give 11 (2.42 g, 100% ); [α]_{D}+12.6° (c 1.0, CHCl₃); ¹H NMR (600 MHz, CDCl₃), δ=7.15-7.44 (m, 45 H, Ar), 5.79-5.85 (m, 1 H, OCH₂CH═CH₂), 5.19-5.22 (m, 1 H, OCH₂CH═CH₂), 5.11-5.13 (m, 2 H, 1a-H, OCH₂CH═CH₂), 4.95 (s, 1 H, 1b-H), 4.87 (m, 2 H, CH₂Ph), 4.77 (d, ²J=11.4 Hz, 1 H, 1/2 CH₂Ph), 4.44-4.73 (m, 14 H, 7 CH₂Ph), 4.35 (d, ²J=11.4 Hz, 1 H, 1/2 CH₂Ph), 4.23 (m, 1 H, 1c-H), 4.13 (m, 1 H, 2a-B), 4.09 (m, 1 H, OCH₂CH═CH₂), 4.04 (m, 1 H, 2b-H), 4.0 (dd, ³J=3.0 Hz, 6.6 Hz, 1 H, 3 a-H), 3.74-3.93 (m, 8 H, 3b-H, 4a-H, 4b-H, OCH₂CH═CH₂, 5a-H, 6b-H, 6′b-H, 6a-H), 3.58-3.72 (m, 4 H, 5b-H, 640 a-H, 6c-H, 6′c-H), 3.42-3.52 (m, 3 H, 3c-H, 4c-H, 2c-H), 3.36 (m, 1 H, 5c-H); ¹³C-NMR (125 MHz, CDCl₃), 139.0-138.1, 133.9, 128.7-127.4, 117.0, 107.7, 100.5, 98.3, 84.2, 79.9, 77.5, 77.4, 77.3, 77.2, 77.0, 76.9, 76.8, 75.6, 75.3, 75.2, 75.1, 74.9, 74.7, 74.1, 73.5, 73.3, 73.2, 72.5, 72.4, 71.9, 69.6, 69.3, 67.9; ES HRMS calcd. for C₈₄H₉₀O₁₆Na 1377.612658, found 1377.612341.

### Example 9

### Allyl (3,4,6-tri-O-benzyl-β-D-mannopyranosyl)-(1→2)-(3,4,6-tri-O-benzyl-α-D-mannopyranosyl)-(1→2)-3,4,6-tri-O-benzyl-α-D-mannopyranoside (12)

Allyl trisaccharide (1.41 g) 11 was oxidized and the keto derivative was reduced as outlined above to give 12 (1.16 g, 82%): [α]_{D}+1.20 (c 1.0, CHC₃); ¹H NMR (600 MHz, CDCl₃), δ=7.11-7.39 (m, 45 H, Ar), 5.79-5.85 (m, 1 H, OCH₂CH═CH₂), 5.19-5.22 (m, 1 H, OCH₂CH═CH₂), 5.12-5.14 (m, 2 H, 1a-H, OCH₂CH═CH₂), 4.91-4.93 (m, 2 H, 1b-H, 1/2 CH₂Ph), 4.49-4.85 (m, 13 H, 13/2 CH₂Ph), 4.42 (m, 3 H, 2a-H, CH₂Ph), 4.34-4.36 (m, 3 H, 1c-H, CH₂Ph), 4.12 (m, 1 H, 2b-H), 4.07 (m, 1 H, OCH₂CH═CH₂), 4.02 (d, ³J=1.8 Hz, 1 H, 2c-H), 3.91-3.94 (m, 4 H, 3a-H, 3b-H, 6b-H, 6′b-H), 3.87 (t, ³J=9.0 Hz, 1 H, 4c-B), 3.83 (m, 1H, OCH₂CH═CH₂), 3.64-3.78 (m, 7 H, 4a-H, 4b-H, 5b-H, 5a-H, 6a-H, 6′a-H, 6c-H), 3.59 (m, 1 H, 6′c-H), 3.33 (d, ³J=9.0 Hz, 1 H, 3c-H), 3.19 (m, 1 H, 5c-H); ¹³C-NMR (125 MHz, CDCl₃), 138.7-138.1, 133.9, 128.5-127.4, 117.2, 99.6, 98.2, 97.3, 81.0, 80.3, 77.3, 75.2, 75.1, 75.0, 74.8, 74.7, 74.5, 74.3, 74.1, 73.4, 73.3, 73.2, 72.8, 72.0, 71.9, 71.6, 71.1, 70.8, 69.5, 69.3, 67.9, 67.8; ES HRMS calcd. for C₈₄H₉₀O₁₆Na 1377.612658, found 1377.612657.

### Example 10

### Allyl (2-O-acetyl-3,4,6-tri-O-benzyl-β-D-glucopyranosyl)-(1→2)-(3,4,6-tri-O-benzyl-β-D-mannopyranosyl)-(1→2)-(3,4,6-tri-O-benzyl-α-D-mannopyranosyl)-(1→2)-3,4,6-tri-O-benzyl-α-D-mannopyranoside (13)

Allyl trisaccharide 12 (1.08 g) was glycosylated as outlined above to give 13 (1.08 g, 74%): [α]_{D}−10.3° (c 1.0, CHCl₃); ¹H NMR (600 MHz, CDCl₃), δ=7.04-7.40 (m, 60 H, Ar), 5.78-5.84 (m, 1 H, OCH₂CH═CH₂), 5.17-5.20 (m, 1 H, OCH₂CH═CH₂), 5.15 (t, ³J=9.0 Hz, 1 H, 2d-H), 5.06-5.10 (m, 2 H, 1d-H, OCH₂CH═CH₂), 4.99 (s, 1 H, 1a-H), 4.95 (d, ²J=11.4 Hz, 1 H, 1/2 CH₂Ph), 4.76-4.91 (m, 6 H, 1c-H, 1b-H, 2 CH₂Ph), 4.41-4.69 (m, 18 H, 9 CH₂Ph), 4.37 (d, ²J=11.4 Hz, 1 H, 1/2 CH₂Ph), 4.22 (br, 1 H, 2a-H), 4.18 (d, ³J=3.0 Hz, 1 H, 2b-H), 4.07 (m, 1 H, OCH₂CH═CH₂), 4.04 (m, 1 H, 2c-H), 3.77-3.95 (m, 6 H, OCH₂CH═CH₂, 3d-H, 5a-H, 3a-H, 3c-H, 6c-H), 3.55-3.75 (m, 13 H, 4a-H, 4b-H, 4c-H, 4d-H, 5c-H, 5d-H, 5b-H, 6a-H, 6′a-H, 6′c-H, 6d-H, 6b-H, 6′b-H), 3.26-3.31 (m, 2 H, 6′d-H, 3b-H); ¹³C-NMR (125 MHz, CDCl₃), 138.8-137.9, 133.8, 128.6-127.3, 117.2, 101.0, 99.5, 98.0, 83.5, 79.9, 78.2, 78.1, 76.8, 75.4, 75.2, 75.1, 75.0, 74.9, 74.8, 74.7, 74.5, 74.4, 73.5, 73.3, 73.2, 72.7, 72.3, 71.8, 71.7, 70.5, 69.6, 69.5, 69.3, 69.2, 69.1, 67.8; ES HRMS calcd. for C₁₁₃H₁₂₀O₂₂Na 1851.816897, found 1851.817052.

### Example 11

### Allyl (3,4,6-tri-O-benzyl-β-D-glucopyranosyl)-(1→2)-(3,4,tri-O-benzyl-β-D-mannopyranosyl)-(1→2)-(3,4,6-tri-O-benzyl-α-D-mannopyranosyl)-(1→2)-3,4,6-tri-O-benzyl-α-D-mannopyranoside (14)

Allyl tetrasaccharide 13 (1.08 g) was deacetylated as outlined above to give 14 (1.05 g, 100% ): [α]_{D}−26° (c 1.0, CHCl₃); ¹H NMR (600 MHz, CDCl₃), δ=6.93-7.42 (m, 60 H, Ar), 5.85-5.92 (m, 1 H, OCH₂CH═CH₂), 5.26-5.29 (m, 1 H, OCH₂CH═CH₂), 5.18-5.20 (m, 1 H, OCH₂CH═CH₂), 5.15 (d, J_{1,2}=1.8 Hz, 1 H, 1b-H), 5.07 (d, ²J=11.4 Hz, 1 H, 1/2 CH₂Ph), 4.86-5.02 (m, 7 H, 1a-H, 3 CH₂Ph), 4.74-4.77 (m, 2 H, 2 CH₂Ph), 4.68 (d, J_{1,2}=7.8 Hz, 1 H, 1d-H), 4.67 (d, ²J=11.4 Hz, 1 H, 1/2 CH₂Ph), 4.53-4.64 (m, 8 H, 4 CH₂Ph), 4.31-4.48 (m, 5 H, 2b-H, 2 CH₂Ph), 4.14-4.20 (m, 3 H, 2c-H, 2 a-H, 4b-H), 4.04 (s, 1 H, 1c-H), 3.87-3.97 (m, 5 H, 5b-H, 3a-H, 3b-H, 6d-H, 6′d-H), 3.78-3.82 (m, 4 H, 4c-H, 4a-H, 2d-H, 6′a-B), 3.63-3.74 (m, 7 H, 5a-H, 3d-H, 6b-H, 6′b-H, 6c-H, 6′c-H, 6′a-H), 3.59 (m, 1 H, 5d-H), 3.55 (t, ³J=8.4 Hz, 1 H, 4d-H), 3.28 (dd, ³J=3.6 Hz, 9.6 Hz, 1 H, 3c-H), 3.07 (m, 1 H, 5c-H); ¹³C-NMR (125 MHz, CDCl₃), 139.1-137.9, 133.7, 128.6-127.2, 117.4, 105.3 (¹J_{C-H}=165 Hz), 99.2 (¹J_{C-H}=170 Hz), 97.98 (¹ J_{C-H}=170 Hz), 97.8 (¹J_{C-H}=160 Hz), 86.6, 80.4, 78.9, 77.9, 77.3, 76.9, 76.8, 75.3, 75.2, 75.1, 74.9, 74.8, 74.7, 74.6, 74.2, 74.0, 73.7, 73.4, 73.3, 73.2, 73.1, 71.9, 71.7, 70.6, 69.8, 69.7, 69.4, 69.3, 69.1, 67.8; ES HRMS calcd. for C₁₁₁H₁₁₈O₂₁Na 1809.806332, found 1809.806374.

### Example 12

### Synthesis of Tetrasaccharide (15)

Glycosylation of trisaccharide 12 with donor 2 in the presence of TMSOTf (0.02 equivalents) in CH₂Cl₂ at −10° C. gave the required tetrasaccharide 13 in good yield. Subsequent deacetylation in a mixed solvent of CH₂Cl₂ and MeOH (1:1) gave the β-glucopyrannosyl alcohol 14. Final oxidation and reduction as above afforded the desired tetrasaccharide 15 in 80% yield. The β-(1→2)-mannopyranosyl trisaccharide 8 and tetrasaccharide 15 were obtained on a gram scale.

### Example 13

### 3-(2-Aminoethylthio)-propyl (β-D-mannopyranosyl)-(1→2)-(β-D-mannopyranosyl)-(1→2)-(α-D-mannopyranosyl)-(1→2)-α-D-mannopyranoside (18)

Compound 15 (80 mg, 0.044 mmol) was reacted with 2-aminoethanethiol (250 mg, 2.2 mmol) under UV condition and subsequent debenzylation under Birch condition as reported by literature^{18b} to give free amine 18 (27.5 mg, 80%). ¹H NMR (600 MHz, D₂O), δ=5.14 (m, 1 H, 1b-H), 5.07 (s, 1 H, 1a-H), 4.87 (s, 1 H, 1c-B), 4.85 (s, 1 H, 1d-B), 4.27 (m, 2 H, 2b-H, 2c-H), 4.15 (d, ³J=3.0 Hz, 1 H, 2d-H), 3.97 (m, 1 H, 2a-H), 3.54-3.93 (m, 20 H, 3a-H, 3b-H, 3c-H, 3d-H, 4a-H, 4b-H, 4c-H, 4d-H, 5a-H, 5b-H, 6a-H, 6′a-H, 6b-H, 6′b-H, 6c-H, 6′c-H, 6d-H, 6′d-H, OCH₂CH₂), 3.35-3.39 (m, 2 H, 5c-H, 5d-H), 3.21 (t, ³J=7.2 Hz, 1H, CH₂NH₂), 2.66-2.68 (m, 4 H, SCH₂CH₂NH₂, OCH₂CH₂CH₂S), 1.92 (m, 2 H, OCH₂CH₂CH₂S); ¹³C NMR (125 MHz, D₂O), 101.8 (^{1J}_{C-H}32 162 Hz, C-1d), 101.1 (¹J_{C-H}=174 Hz, C-1b), 99.8 (¹J_{C-H}=156 Hz, C-1c), 99.1 (¹J_{C-H}32 168 Hz, C-1a), 79.6, 79.4, 78.6, 77.2, 74.2, 73.8, 73.7, 73.0, 71.3, 71.1, 70.2, 68.1, 67.8, 67.6, 67.1, 62.0, 61.8, 61.6, 61.5; EMS Caculd. for C₂₉H₅₃NO2₁SH⁺784.3, found 784.4.

### Example 14

### 7-Aza-8,13-dioxo-13-(4-nitro-phenoxy)-4-thia-tridecanyl (β-D-mannopyranosyl)-(1→2)-β-D-mannopyranoside (20)

To a solution of free amine 16 (10 mg, 0.022 mmol) in dry DMF (1 mL) was added diester 19 (42 mg, 0.11 mmol) under argon, and stirred for 5.0 h when TLC indicated almost complete reaction of free amine. Finally, the reaction mixture was co-evaporated with toluene to remove DMF, and the residue was dissolved in CH₂Cl₂ (10 mL), and washed with H₂O (10 mL) containing 1% acetic acid. The water solution was then passed through a C18-Sep-Pac cartridge and eluted with methanol containing 1% acetic acid, to remove any compound that would be irreversibly absorbed to the reverse phase silica column. The solution was concentrated at low pressure to afford crude product as a solid. Final purification on reverse phase silica (C18) was accomplished with a water methanol mixture containing 1% acetic acid gradient to yield pure half ester 20 (9.8 mg, 64%). ¹H-NMR (600 MHz, CD₃OD), δ=8.28 (m, 2 H, C₆H₂), 7.38 (m, 2 H, C₆H₂), 4.78 (s, 1 H, 1b-H), 4.56 (s, 1 H, 1a-H), 4.11 (d, ³J=3.0 Hz, 1 H, 2a-H), 3.98 (m, 2 H, 2b-H, OCH₂CH₂), 3.86 (m, 2 H, 6a-H, 6b-H), 3.60-3.72 (m, 3 H, OCH₂CH₂CH₂, 6′a-H, 6′b-H), 3.49-3.54 (m, 2 H, 4a-H, 4b-H), 3.44-3.46 (dd, ³J=3.5 Hz, 9.5 Hz, 1 H, 3a-H), 3.39-3.42 (dd, ³J=3.5 Hz, 9.5 Hz, 1 H, 3b-H), 3.37 (t, ³J=3.5 Hz, 2 H, CH₂COO), 3.17-3.21 (m, 2 H, 5a-H, 5b-H), 2.60-2.69 (m, 6 H, NHCOCH₂, CH₂CH₂NH, COCH₂CH₂), 2.26 (t, 2 H, SCH₂CH₂NH), 1.86 (m, 2 H, OCH₂CH₂CH₂S), 1.75 (m, 4 H, CH₂CH₂CH₂CH₂); EMS Calcd. C₂₉H₄₄N₂O₁₆SNa 731.24, found 731.2.

### Example 15

### 7-Aza-8,13-dioxo-13-(4-nitro-phenoxy)-4-thia-tridecanyl (β-D-mannopyranosyl)-(1→2)-(β-D-mannopyranosyl)-(1→2)-β-D-mannopyranoside (21).

Free amine 17 (12.5 mg, 0.02 mmol) was reacted with diester 19 (40 mg, 0.1 mmol) as above outlined to give the half ester 21 (13 mg, 75%). ¹H-NMR (600 MHz, CD3OD), δ=8.28 (m, 2 H, C₆H₂), 7.38 (m, 2 H, C₆H₂), 4.94 (s, 1 H, 1c-H), 4.78 (s, 1 H, 1b-H), 4.56 (s, 1 H, 1a-H), 4.22 (d, ³J=3.0 Hz, 1 H, 2b-H), 4.04 (m, 2 H, 2a-H, 2c-H), 4.0 (m, 1 H, OCH₂CH₂), 3.97-3.99 (m, 3 H, 6a-H, 6b-H, 6c-H), 3.62-3.88 (m, 4 H, 6′a-H, 6′b-H, 6′c-H, OCH₂CH₂), 3.41-3.55 (m, 6 H, 3a-H, 3b-H, 3c-H, 4a-H, 4b-H, 4c-H), 3.34-3.38 (m, 2 H, CH₂COO), 3.17-3.31 (m, 3 H, 5a-H, 5b-H, 5c-H), 2.62-2.67 (m, 6 H, NHCOCH₂, CH₂CH₂NH, COCH₂CH₂), 2.26 (t, 2 H, SCH₂CH₂NH), 1.87-1.97 (m, 2 H, OCH₂CH₂CH₂S), 1.72-1.78 (m, 4H, CH₂CH₂CH₂CH₂); EMS Calcd. C₃₅H₅₄N₂O₂₁SNa 893.29, found 893.3.

### Example 17

### 7-Aza-8,13-dioxo-13-(4-nitro-phenoxy)-4-thia-tridecanyl (β-D-mannopyranosyl)-(1→2)-(β-D-mannopyranosyl)-(1→2)-(α-D-mannopyranosyl)-(1→2)-a-D-mannopyranoside (22)

Free amine 18 (9 mg, 0.01 mmol) was reacted with diester 19 (22 mg, 0.05 mmol) as above outlined to give the half ester 22 (7 mg, 62%). ¹H-NMR (600 MHz, CD₃OD), δ8=8.31 (m, 2 H, C₆H₂), 7.38 (m, 2 H, C₆H₂), 5.05 (d, 1 H 1b-H), 5.02 (d, 1 H, 1a-H), 4.82 (s, 1 H, 1c-H), 4.73 (s, 1 H, 1d-H), 4.16 (m, 1 H, 2b-H), 4.12 (d, ³J=3.0 Hz, 1 H, 2d-H), 4.04 (d, ³J=3.0 Hz, 1 H, 2c-H), 3.40-3.88 (m, 21 H, 2a-H, 3a-H, 4a-H, 5a-H, 6a-H, 6′a-H, 3b-H, 4b-H, 6b-H, 6′b-H, 3c-H, 4c-H, 5c-H, 6c-H, 6′c-H, 3d-H, 4d-H, 6d-H, 6′d-H, OCH₂CH₂CH₂S), 3.36 (t, 2 H, CH₂NHCO), 3.23 (m, 2 H, 5b-H, 5d-H), 2.64-2.67 (m, 6 H, CH₂CH₂S, SCH₂CH₂NH, NHCOCH₂), 2.28 (t, 2 H, CH₂COO), 1.73-1.86 (m, 6 H, OCH₂CH₂CH₂S, OCCH₂CH₂CH₂CH₂CO); EMS Calcd. For C₄₁H₆₄N₂O₂₆SNa 1055.2, found 1055.3.

### Example 18

### Glycoconjugates

The general procedure for generating protein-carbohydrate conjugates was as followed. BSA (10 mg) was dissolved in phosphate buffer pH 7.5 (2 ml), and the half ester was dissolved in DMF (100 μl), then the solution was injected into the reaction medium slowly, and the reaction was left for one day at room temperature. The mixture was then diluted with deionized water and dialysed against 5 changes of deionized water (2 l) or started as a PBS solution pH=7.2 for tetanus toxoid (TT) conjugates. The solution was lyophilized to a white solid.

TABLE 3
BSA and TT Mannopyranan conjugates

| | Saccharide | Molar ratio of | hapten | Incorporation |
|---|---|---|---|---|
| Conjugate | (mg) | protein:monoester | incorporated | efficiency (%) |
| 23 | 1 | 1:20 | 7.3 | 36.6 |
| 24 | 1.6 | 1:20 | 48.8 | 44 |
| 25 | 2.3 | 1:30 | 12.2 | 40.7 |
| 26 | 3 | 1:40 | 13 | 32.5 |
| 27 | 2.6 | 1:30 | 12.6 | 42 |

### Example 19

### Pentenyl (3,4,6-tri-O-benzyl-2-O-acetyl-β-D-glucopyranosyl)-(1→2)-3,4,6-tri-O-benzyl-β-D-mannopyranoside (29)

The procedure used was analogous to the preparation of Allyl (3,4,6-tri-O-benzyl-2-O-acetyl-β-D-glucopyranosyl)-(1→2)-3,4,6-tri-O-benzyl-β-D-mannopyranoside, and used glycosyl donor 2 (650 mg, 1.02 mmol), acceptor 28 [Rodebaugh, R.; Debenham, J. S.; Fraser-Reid; Snyder, J. P.; J. Org. Chem., 1999, 64, 1758-1761] (440 mg 0.85 mmol), dichloromethane (8 mL), trimethylsilyl trifluorometanesulfonate (6 μl) and activated 4 Å molecular sieves (100 mg). Column chromatography in n-hexane/ethyl acetate (4:1) gave the disaccharide 29 (758 mg, 85%).

¹H-NMR (CDCl₃, 600 MHz): 7.2-7.42 (m, 30 H, Ar), 5.92-5.85 (m, 1 H, CH₂CH═CH₂), 5.17-5.14 (dd, ³J=7.8 Hz, 9.6 Hz, 1 H, 2b-H), 5.7-5.11 (m, 1 H, CH₂CH═CHₐ), 5.02-5.04 (m, 1 H, CH₂CH═CH_{b}), 4.76-4.97 (m, 6 H, 1b-H, 5/2 CH₂Ph), 4.48-4.6 (m, 7 H, 7/2 CH₂Ph), 4.33 (s, 1 H, 1a-H), 4.28 (d, ²J=3.0 Hz, 1 H, 2a-H), 3.89-3.93 (m, 1 H, OCHa), 3.79 (m, 2 H, 6a-H, 6′a-H), 3.75 (dd, ³J=8.4 Hz, 1 H, 3b-H), 3.69 (m, 1 H, 6b-H), 3.58-3.65 (m, 4 H, 4a-H, 4b-H, 5b-H, 6′b-H), 3.44-3.51 (m, 3 H, 3a-H, 5a-H, OCHb), 2.25 (m, 2 H, CH₂CH═CH₂), 1.98 (s, 3 H, Ac), 1.78 (m, 2 H, OCH₂CH₂); EMS Caculd. for C₆₁H₆₈O₁₂Na⁺1015.46030, found 1015.46036.

### Example 20

### Pentenyl (3,4,6-tri-O-benzyl-β-D-glucopyranosyl)-(1→2)-3,4,6-tri-O-benzyl-β-D-mannopyranoside (30)

The procedure used was analogous to the preparation of Allyl (3,4,6-tri-O-benzyl-β-D-glucopyranosyl)-(1→2)-3,4,6-tri-O-benzyl-β-D-mannopyranoside, and used disaccharide 29 (758 mg, 0.76 mmol), sodium methoxide (5 mg), dichloromethane (5 mL), methanol (5 mL). Column chromatography in n-hexane/ethyl acetate (4:1) gave the disaccharide 30 (722 mg, 100% ).

¹H-NMR (CDCl₃, 600 MHz): 7.2-7.42 (m, 30 H, Ar), 5.92-5.85 (m, 1 H, CH₂CH═CH₂), 5.07-5.11 (m, 2 H, CH₂CH═CHₐ, 1/2 CH₂Ph), 5.0-5.03 (m, 1 H, CH₂CH═CH_{b}), 4.90-4.98 (m, 3 H, 3/2 CH₂Ph), 4.83 (d, ²J=11.4 Hz, 1 H, 1/2 CH2Ph), 4.73 (d, ³J=7.8 Hz, 1 H, 1b-H), 4.66 (d, ²J=12.0 Hz, 1 H, 1/2 CH₂Ph), 4.49-4.61 (m, 6 H, 3 CH₂Ph), 4.41 (s, 1-H, 1a-H), 4.28 (d, ³J=3.0 Hz, 1 H, 2a-H), 3.96-4.0 (m, 1 H, OCHa), 3.93 (t, ³J=9.6 Hz, 1 H, 4a-H), 3.75-3.82 (m, 4 H, 2b-H, 6b-H, 6a-H, 6′a-H), 3.68-3.71 (m, 2 H, 3b-H, 6′b-H), 3.49-3.62 (m, 4 H, 5b-H, 3a-H, 4b-H, OCHb), 3.43 (m, 1 H, 5a-H), 2.20 (m, 2 H, CH₂CH═CH₂), 1.78 (m, 2 H, OCH₂CH₂); ¹³C-NMR (125 MHz, CDCl₃), 139.1-115.1, 104.1 (¹J_{C-H}=162 Hz), 100.5 (¹J_{C-H}=156 Hz), 85.2, 80.3, 76.8, 75.7, 75.4, 75.3, 75.1, 74.8, 74.7, 73.4, 70.0, 69.8, 69.3, 69.2; EMS Caculd. for C₅₉H₆₆O₁₁Na⁺973.44974, found 973.44977.

### Example 21

### Pentenyl (3,4,6-tri-O-benzyl-β-D-mannopyranosyl)-(1→2)-3,4,6-tri-O-benzyl-β-D-mannopyranoside (31)

The procedure used was analogous to the preparation of Allyl (3,4,6-tri-O-benzyl-β-D-mannopyranosyl)-(1→2)-3,4,6-tri-O-benzyl-β-D-mannopyranoside 5, and used disaccharide 30 (630 mg, 0.66 mmol), dimethyl sulfoxide (10 mL), acetic anhydride (5 mL), THF (10 mL), L-selectride (1 M, 2 mL). Column chromatography in n-Hexane/ethyl acetate (5:2) gave the disaccharide 31 (504 mg, 80%).

¹H-NMR (CDCl₃, 600 MHz): 7.19-7.42 (m, 30 H, 5.92-5.85 (m, 1 H, CH₂CH═CH₂), 4.99-5.03 (m, 1 H, CH₂CH═CHₐ), 4.93-4.98 (m, 4 H, 1b-H, CH₂Ph, CH₂CH═CH_{b}),4.84-4.90 (m, ²J=12.0 Hz, CH₂Ph), 4.56-4.69 (m, 4 H, 2 CH₂Ph), 4.44-4.50 (m, 5 H, 2a-H, 2 CH₂Ph), 4.38 (s, 1 H, 1a-H), 4.34 (dd, ³J=1.2 Hz, 3.0 Hz, 1 H, 2b-H), 3.92-3.94 (m, 2 H, 4b-H, OCHₐ), 3.77-3.80 (m, 3 H, 4a-H, 6a-H, 6b-H), 3.67-3.74 (m, 2 H, 6′a-H, 6′b-H), 3.56-3.59 (m, 2 H, 3a-H, 3b-H), 3.49-3.52 (m, 1 H, 5b-H), 3.42-3.47 (m, 2 H, 5a-H, OCHb), 2.20 (m, 2 H, CH₂CH═CH₂), 1.78 (m, 2 H, OCH₂CH₂); ¹³C-NMR (125 MHz, CDCl₃), 138.4-115.1, 101.1 (¹J_{C-H}=168 Hz), 99.3 (¹J_{C-H}32 156 Hz), 81.5, 80.4, 75.6, 75.1, 74.4, 74.2, 73.5, 73.3, 70.8, 70.7, 70.1, 69.9, 69.5, 69.2, 67.7; EMS Caculd. for C₅₉H₆₆O₁₁Na⁺973.44974, found 973.44991.

### Example 22

### Pentenyl (2,3,4,6-tetra-O-acetyl-β-D-mannopyranosyl)-(1→2)-3,4,6-tri-O-acetyl-β-D-mannopyranoside (32)

Subsequent debenzylation of disaccharide 31 (80 mg, 0.084 mmol), under Birch conditions, THF (2 mL), t-butanol (2 mL), Na (100 mg) as reported in the literature gave a crude product, which was acetylated in pyridine:acetic anhydride (4 mL of a 1:1 mixture) to afford pure compound 32 (35 mg, 60%).

¹H-NMR (CDCl₃, 600 MHz): 5.76-5.84 (m, 1 H, CH₂CH═CH₂), 5.53 (dd, ³J=1.2 Hz, 3.6 Hz, 1 H, 2b-H), 5.03-5.21 (m, 2 H, 4a-H, 4b-H), 4.95-5.03 (m, 3 H, 3b-H, CH₂CH═CH₂), 4.85 (s, 1 H, 1b-H), 4.63 (dd, ³J=3.2 Hz, 10.1 Hz, 1 H, 3a-H), 4.47 (s, 1 H, 1a-H), 4.35 (d, ³J=3.0 Hz, 1H, 2a-H), 4.19-4.29 (m, 2 H, 6a-H, 6b-H), 4.06-4.10 (m, 1 H, 6′a-H), 3.98 (m, 1 H, 6′b-H), 3.89 (m, 1 H, OCHa), 3.56 (m, 1 H, 5b-H), 3.51 (m, 1 H, 5a-H), 3.41-3.48 (m, 1 H, OCHb), 1.99-2.2 (m, 23 H, CH₂CH═CH₂, 7 Ac), 1.78 (m, 2 H, OCH₂CH₂); ¹³C-NMR (125 MHz, CDCl₃), 169.2-170.8, 137.8, 115.0, 99.8 (¹J_{C-H=168} Hz), 97.9 (¹J_{C-H}=156 Hz), 72.3, 72.1, 71.9, 71.8, 70.7, 69.4, 68.6, 66.3, 65.1, 62.5, 61.9; EMS Caculd. for C₃₁H₄₄O₁₈Na⁺727.24199, found 727.24186.

### Example 23

### 1-Thioacetyl-pentyl (2,3,4,6-tetra-O-acetyl-β-D-mannopyranosyl)-(1→2)-3,4,6-tri-O-acetyl-β-D-mannopyranoside (33)

Compound 32 (53 mg, 0.075 mmol) was dissolved in CH₂Cl₂ (4 mL), and HSCOCH₃ (18 μl, 0.226 mmol), was added, then the reaction mixture was bubbled with with argon for 2 min. The reaction was performed under UV condition. After 15 min., NMR indicated that the starting material had disappeared. The mixture was diluted with CH₂C1₂ (10 mL), washed with saturated sodium bicarbonate solution, brine, and then dried over MgSO₄. Finally, the organic phase was evaporated to give the crude. The residue was purified by chromatography to afford pure compound 33 (51 mg, 87%).

¹H-NMR (CDCl₃, 600 MHz): 5.54 (d, ³J=3.0 Hz, 1 H, 2b-H), 5.18-5.21 (m, 2 H, 4a-H, 4b-H), 5.05 (m, 1 H, 3b-H), 4.85 (s, 1 H, 1b-H), 4.64 (dd, ³J=10.2 Hz, 3.0 Hz, 1 H, 3a-H), 4.47 (s, 1 H, 1a-H), 4.35 (d, ³J=3.0 Hz, 1 H, 2a-H), 4.29 (m, 1 H, 6b-H), 4.22 (m, 1 H, 6a-H), 4.09 (m, 1 H, 6′a-H), 4.01 (m, 1 H, 6′b-H), 3.87 (m, 1 H, OCHa), 3.61 (m, 1 H, 5b-H), 3.50 (m, 1 H, 5a-H), 3.42 (m, 1 H, OCHb), 2.87 (t, 2 H, CH₂SAc), 2.30 (s, 3 H, SAc), 1.98-2.2 (m, 21 H, 7 Ac), 153-1.70 (m, 4 H, OCH₂CH₂CH₂CH₂CH₂SAc), 1.4 (m, 2 H, OCH₂CH₂CH₂CH₂CH₂SAc); ¹³C-NMR (125 MHz, CDCl₃), EMS Caculd. for C₃₃H₄₈O₁₉Na⁺803.24027, found 803.24013.

### Example 24

### (2-[2-(2-azidoethoxy)ethoxy]ethyl)-2,3,4,6-tetra-O-allyl-D-glucopyranoside (35)

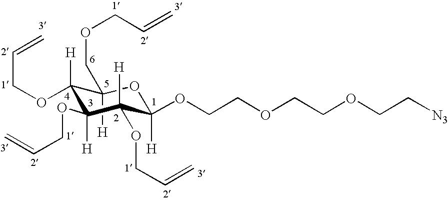

To a stirred solution of compound 34 [Kitov, P. I., Tsvetkov, Yu. E, Bakinovsky, L. V. Doki. Chem. (Engl Transl.) 1993, 329, 1-3] (2 mmol) in THF (20 ml) was added allylbromide (1.69 ml, 20 mmol) and NaH (560 mg of NaH 60%, 14 mmol) by small portions. At end of addition, the reaction was put under reflux for 90 minutes and then cooled to room temperature. Water (5 mL) was added dropwise to destroy excess of NaH. The translucid solution was extracted with AcOEt (1*100 ml, 3*30 ml). The combined organic layers were dried (MgSO4) and solvents were evaporated. The residue (1.256 g) was chromatographied on silicagel (acetone/hexane (1:4), Rf=0.32) to give 35 (648 mg, 65% from 34) as a colorless oil.

¹H-NMR (C₆D₆, 500 MHz): 5.995 (2*dddd (=ddt), H(C2′)a+H(C2′)b, 3J3′trans=17.2, 3J3′cis=10.5, 3J1′=5.4, 3J1′=5.4); 5.90 (dddd (=ddt), H(C2′)c, 3J3′trans=17.2, 3J3′cis=10.6, 3J1′=5.4, 3J1′=5.4); 5.83 (dddd (=ddt), H(C2′)d, 3J3′trans=17.2, 3J3′cis=10.6, 3J1′=5.4, 3J1′=5.4); 5.32 (2*dm, H(C3′)transa+H(C3′)transb, 3J2′=17.2, Jm=1.8); 5.25 (2*dm shifted of Jm, H(C3′)transc+H(C3′)transd, 3J2′=17.2, Jm=1.9); 5.07 (2*dm, H(C3′)cisa+H(C3′)cisb, 3J2′=10.5, 3J=1.6); 5.03 (2*dm, H(C3′)cisc+H(C3′)cisd, 3J2′=10.6, 3J=1.7); 4.52 (dddd (=ddte), H(C1′)a, 2J=12.9, 3J2′=5.4, 4J3′=1.6, 4J3′=1.6); 4.48 (dddd (=ddte), H(C1′)b, 2J=12.9, 3J2′=5.4, 4J3′=1.6, 4J3′=1.6); 4.36 (dddd (=ddte), H(C1′)c, 2J=12.8, 3J2′=5.4, 4J3′=1.5, 4J3′=1.5); 4.36 (dddd (=ddte), H(C1′)b, 2J=12.9, 3J2′=5.4, 4J3′=1.5, 4J3′=1.5); 4.29 (d, H(C1), 3J2=7.7); 4.23 (dddd (=ddte), H(C1′)a, 2J=12.9, 3J2′=5.4, 4J3′=1.5, 4J3′=1.5); 4.12 (dddd (=ddte), H(C1′)c, 2J=12.8, 3J2′=5.4, 4J3′=1.5, 4J3′=1.5); 3.93 (dddd (=ddte), H(C1′)d, 2J=13.2, 3J2′=5.3, 4J3′=1.5, 4J3′=1.5); 3.90 (ddd (=dt), H(OCH2CH2O), 2J=11.1, 3J=5.3, 3J=5.3); 3.87 (dddd (=ddte), H(C1′)d, 2J=13.2, 3J2′=5.4, 4J3′=1.5, 4J3′=1.5); 3.63 (d, 2H(C6), 3J5=3.3); 3.59 (ddd, H(OCH2CH2O), 2J=10.9, 3J=6.9, 3J=4.0); 3.53-3.44 (m, H(C3)+H(*C4)+H(OCH2CH2O)); 3.43-3.37 (m, H(OCH2CH2O)+H(C2)+2H(OCH2CH2O)); 3.33-3.27 (m, 2H(OCH2CH2O)+H(C5)); 3.175 (dd (=t), 2H(OCH2CH2N3)); 2.77 (dd (=t), 2H(OCH2CH2N3)); ¹³C-NMR (C6D6, 125 MHz): 136.3+136.3+135.9+135.5 (4C, 4*C2′); 116.2+115.8+115.8+115.6 (4C, 4*C3′); 104.1 (C1); 84.8 (C3 or C4); 82.3 (C2); 77.9 (C4 or C3); 75.4 (C(5)); 74.4 (C1′b); 73.7 (C1′a); 73.5 (C1′c); 72.5 (C1′d); 70.9+70.9+70.8 (3C, 3*C(OCH2CH2O)); 70.2 (C(OCH2CH2N3)); 69.6 (C6); 69.0 (C(OCH2CH2O)); 50.7 (C(OCH2CH2N3))

Low resolution mass spectra: 520.2 (100% , M+Na⁺)

Elemental Composition Report for C₂₄H₃₉O₈N₃Na: Calculated=520.26294; Measured=520.26239

MA: Elemental analysis for C₂₄H₃₉O₈N₃: Calculated=57.93% C, 7.90% H, 8.44% N; Found=57.95% C, 8.11% H, 8.5% N

### Example 25

### (2-[2-(2-azidoethoxy)ethoxy]ethyl)-2,3,4,6-tetra-O-(2,3-epoxypropyl)-D-glucopyranoside (36)

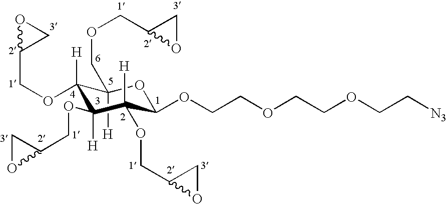

To a stirred solution of 35 (208 mg, 0.42 mmol) in CH₂Cl₂ (2.1 ml) was added, at room temperature, mCPBA (618 mg of 77%, 2.51 mmol, 6 eq) by small portions over 10 minutes. The reaction was stirred at room temperature for 75 minutes (metachlorobenzoic acid precipitated as a white solid). CH₂Cl₂ (1 ml) was added and the reaction was put under reflux for 105 minutes. The reaction mixture was diluted with CH₂Cl₂ (3 ml) and filtrated. The white solid was washed with CH₂Cl₂ (3 ml, 3 times). The combined filtrate were neutralized with a saturated solution of NaHCO₃ (5 ml). The aqueous phase was extracted with CH₂Cl₂ (10 ml, 3 times). The combined organic layers were dried (MgSO₄) and solvents were evaporated. The residue was chromatographed on silica gel (acetone/hexane (1:1.5), R_{f}=0.28) to give 36 (168 mg, 72%) as a colorless oil.

¹H-NMR (CDCl₃, 500 MHz): 4.25 (d, 1H, H(C1), 3J2=7.7); 4.18-4.04 (m, 1H, H(C1′)A), 4.04-3.92 (m, 3H, 2H(C6)+H(C1′)B); 3.92-3.80 (m, 1H, H(C1′)C); 3.80-3.60 (m, 13H, 2H(OCH₂CH₂N₃)+8H(OCH₂CH₂O)+H(C5))+H(C1′)B+H(C1′)D); 3.60-3.52 (m, 1H, H(C1′)A); 3.52-3.44 (m, 1H, H(C1′)D); 3.40-3.30 (m, 5H, 2H(OCH₂CH₂N₃)+H(C3))+H(C4)+H(C1′)C); 3.22-3.10 (m, 5H, 4*H(C2′)+H(C2)); 2.82-2.74 (m, 4H, 4*H(C3′)); 2.62-2.54 (m, 4H, 4*H(C3′)); ¹³C-NMR (CDCl₃, 125 MHz): 103.4-103.3 (C1); 84.9-84.5 (C3); 82.7-82.5 (C2); 78.1-77.5 (C4); 75.0-71.9 (4*C1′); 70.7-69.8 (C(5)+4C(OCH2CH2O)+C(OCH2CH2N3)); 68.9 (C6); 50.9-50.5 (4*C(2′)+C(OCH2CH2N3)); 44.6-44.1 (4*C(3′))

Low resolution mass spectra: 584.2 (100% , M+Na⁺)

Elemental Composition Report for C₂₄H₃₉O₈N₃Na: Calculated=584.24260; Measured: 584.24284

Elemental analysis for C₂₄H₃₉O₈N₃: Calculated=51.33% C, 7.00% H, 7.48% N; Found=51.24% C, 7.06% H, (% N not measured)

### Example 26

### (2-[2-{2-Azidoethoxy}ethoxy]ethyl) 2,3,4,6-tetrakis-(2-hydroxy-[pentyl (β-D-mannopyranosyl)-(1→2)-β-D-mannopyranosyl ]-3-thiapropyl)-β-D-glucopyranoside (37)

To a stirred solution of 33 (47 mg, 0.06 mmol) and 36 (6.8 mg, 0.012 mmol) in MeOH (2 ml) was bubbled argon for 45 minutes at room temperature. K₂CO₃ (12 mg, 0.087 mmol) was added and the solution stirred for 1 hour at room temperature, and the reaction solution became turbid, then five drops of degassed water was added, and stirred at room temperature overnight. The reaction mixture was diluted with MeOH (10 mL) and neutralized with Amberlite (H⁺-Exchanger-Resin). The solution was filtered and the resin was washed with MeOH (5 ml, 3 times). Solvents were evaporated and the residue was purified by HPLC on C18-preparative column [using gradient a) 5 min. H₂O/MeOH (100:0) b) 15 min.→H₂O/MeOH (67:33) c) 60 min.→H₂O/MeOH (0:100) d) 20 min. H₂O/MeOH (0:100)] to give product 37 (17 mg, 60%) as a white powder.

¹H-NMR (D₂O, 600 MHz): 4.84 [s, 4 H, 4 (1b-H)], 4.75 [s, 4 H, 4 (1b-h)], 4.54 (m, 1 H, Glu-1a-H), 4.25 [m, 4 H, 4 (2a-H)], 4.13 [m, 4 H, 4 (2b-B)], 3.35-4.05 [m, 77 H, 4 (3b-H), 4 (4b-H), 4 (5b-H), 4 (6b-H), 4 (6′b-H), 4 (3a-H), 4 (4a-H), 4 (5a-H), 4 (6a-H), 4 (6′a-H), 4 (OCH₂), 4 (OCH), Glu-6a-H, Glu-6′a-H, Glu-2a-H, Glu-3a-H, Glu-4a-H, 3 OCH₂CH₂)], 3.24 (m, 1 H, Glu-5a-H), 2.23-2.8 [m, 16 H, 4 (CH₂SCH₂)], 1.62-1.68 [m, 16 H, 4 (OCH₂CH₂CH₂CH₂CH₂S)], 1.45-1.50 [m, 8 H, 4 (OCH₂CH₂CH₂CH₂CH₂S)]MS (positive mode, DHB, H2O): Caculd. for C₉₂H₁₆₇N₃O₅₆S₄K⁺2376.92 found 2377.33.

### Example 27

### (2-[2-{2-Aminoethoxy}ethoxy]ethyl) 2,3,4,6-tetrakis-(2-hydroxy-[pentyl (β-D-mannopyranosyl)-(1→2)-β-D-mannopyranosyl]-3-thiapropyl)-β-D-glucopyranoside (38)

Compound 37 (29 mg) was dissolved in a mixture solvent of H₂O, pyridine and NEt₃ (10:1:0.3) (10 ml), and H₂S was bubbled in the reaction mixture at room temperature. After 4.0 hours, TLC check indicated that the starting material disappeared. Finally, the mixture solvent was removed to remove the excess H₂S, then the residue was dissolved in H₂O (2 ml), and lyophilization gave crude 38 as white powder.

MALDI-MS (positive mode, DHB, H₂O): Cacld. for C₉₂H₁₆₉NO₅₆S₄Na⁺2334.93 found 2335.64.

### Example 28

### 9-Aza-10,15-dioxo-15-(4-nitro-phenoxy)-(2-[2-{2-ethoxy}ethoxy}ethyl) 2,3,4,6-tetrakis-(2-hydroxy-[pentyl (β-D-mannopyranosyl)-(1→2)-β-D-mannopyranosyl]-3-thiapropyl)-β-D-glucopyranoside (39)

To a solution of free amine 38 (3.5 mg) in dry DMF (1 mL) was added diester 19 (12 mg, 0.03 mmol) under argon, and stirred for 5.0 h when TLC indicated almost complete reaction of free amine. Finally, the reaction mixture was co-evaporated with toluene to remove DMF, and the residue was dissolved in CH₂Cl₂ (5 mL), and washed with H₂O (5 mL) containing 1% acetic acid. The water solution was then passed through a C18-Sep-Pac cartridge and eluted with methanol containing 1% acetic acid, to remove any compound that would be irreversibly absorbed to the reverse phase silica column. The solution was concentrated at low pressure to afford crude product as a solid. Final purification on reverse phase silica (C18) was accomplished with a water methanol mixture containing 1% acetic acid gradient to yield pure half ester 39 (2.5 mg, 64%).

### Example 29

### Glycoconjugates on Clustered Modes

The general procedure for generating protein-carbohydrate conjugates was as followed: BSA (10 mg) was dissolved in phosphate buffer pH 7.5 (2 ml), and the half ester 39 was dissolved in DMF (100 μl), then the solution was injected into the reaction medium slowly, and the reaction was left for one day at room temperature. The mixture was then diluted with deionized water and dialyzed against 5 changes of deionized water (2 l) or started as a PBS solution pH=7.2 for tetanus toxoid (TT) conjugates. The solution was lyophilized to a white solid.

TABLE 4
BSA and TT Cluster conjugates

| | | | Cluster | |
|---|---|---|---|---|
| | Cluster 14 | Molar ratio of | hapten | Incorporation |
| Conjugate | (mg) | protein:monoester | incorporated | efficiency (%) |
| 40-BSA | 4.2 | 1:24 | 5.4 | 22.5 |
| 40-TT | 1.6 | 1:20 | 4.6 | 23 |

MALDI-MS (positive mode, matrix sinapinic acid, H₂O): BSA-cluster conjugate (MW 79368), TT-cluster conjugate (MW 165059).

### Example 30

### Preparation of the glycoconjugate-alum Vaccine

A molar sodium bicarbonate solution (about 3.4 g in 40 ml water) was added to a solution of aluminum potassium sulfate (about 7.6 g in 80 ml water). The precipitate was washed three times with about 150 ml PBS at pH 7.2, being centrifuged at about 5,000 rpm between each wash. The pelleted material from the final spin was resuspended in 12 ml PBS and used to absorb conjugates for vaccine preparation.

The above alum suspension (7 ml) was mixed with the tetanus toxoid glycoconjugate (2.4 mg/ml in PBS) and 80 μl of Thermisal (10 mg/mL) was added. The mixture was gently mixed overnight in an inversion mixer. This suspension contained solution phase glycoconjugate and glycoconjugate absorbed to alum in an about 50:50 ratio and was used to immunize rabbits according to the protocol below. The corresponding control vaccine for use in rabbit protection studies contained only tetanus toxoid absorbed to alum, which was prepared in a similar fashion.

For immunization of mice about 2.08 ml alum suspension was mixed with about 320 μl of tetanus toxoid glycoconjugate stock solution (2.34 mg/ml in PBS) and 24 μl of Thermisal (10 mg/ml) was added. The mixture was gently mixed overnight in an inversion mixer and used directly.

### Example 31

### Immunization of Mice with Glycoconjugates 24 and 25 and Antibody Titrations by ELISA

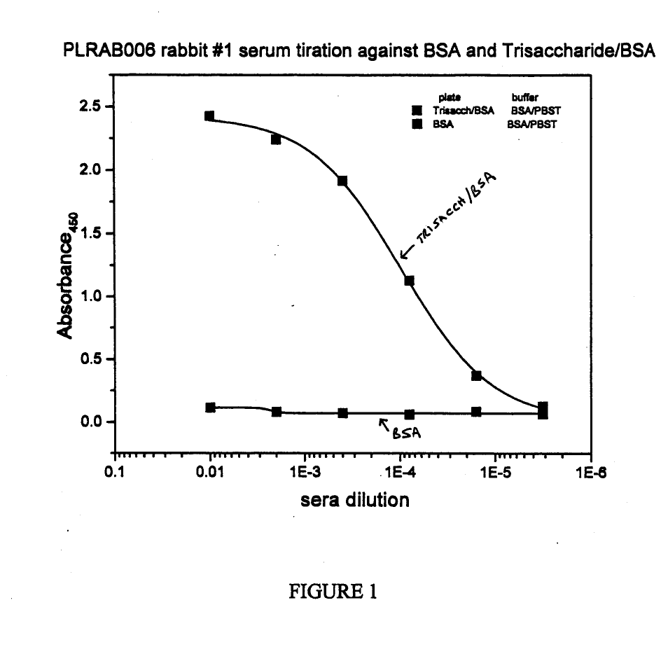

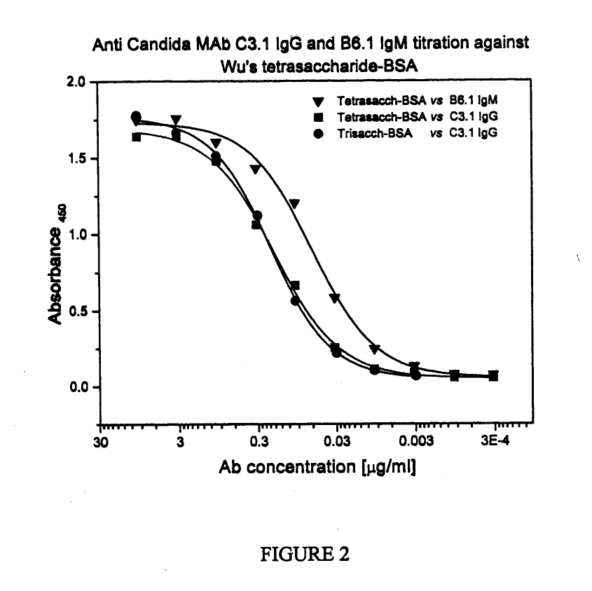

Preliminary experiments established that rabbits immunized with either trisaccharide 26 or tetrasaccharide 27 succeed in raising high titre trisaccharide specific antibodies that bound the corresponding BSA conjugates 24 and 25 but not unconjugated BSA (FIGS. 1 and 2). The same antibodies exhibited comparable titres when ELISA plates were coated with a crude β-mannan cell wall extract from C. albicans. When the three glycoconjugates were used to coat ELISA plates and two protective monoclonal antibodies IgG C3.1 and IgM B6.1 were titred against these antigens all three 23-25 were strongly active. Furthermore it can be seen that trisaccharide and tetrasaccharide conjugates 24 and 25 bound the IgG monoclonal antibody C3.1. In agreement with previously published inhibition data the near identical binding curves strongly suggest that this antibody is largely directed to the common terminal disaccharide present in both structures.

### Example 32

### Immunization of Mice with Glycoconjugates 26 and 27 and Antibody Titrations by ELISA

Groups of 4, 8-10 week-old, Balb/c mice were immunized with glycoconjugates 26 and 27. A total of three injections were given on days 0, 21, and 40. The vaccine was administered over two sites: 200 μL intraperitoneal and 100 μL subcutaneous. Mice were sacrificed on day 47 and serum was collected and frozen after clotting and removal of red cells.

For antibody titration, a 96-well Nunc-lmmuno ELISA plate (MaxiSorp F96) was coated with 100 μl of the BSA oligosaccharide conjugates (5 μg/ml in PBS) and allowed to sit at 4C, overnight. Excess solution was discarded, and the plate was washed 4 times with PBST. Serial ten-fold dilutions of immune sera, starting at 1:10 and ending at 1:10⁶, were assayed together with pre-immune sera diluted 1:1000. The plate was incubated for 2 hours at room temperature and then washed 4 times with PBST. Bound antibody was detected using goat anti-mouse IgG and goat anti-mouse IgM antibodies conjugated to horse radish peroxidase (Kirkegaard and Perry Lab) at a working dilution of 1:2,000 for 1 hour at room temperature. The plate was washed 4 times with PBST. A TMB solution (100 μl) was added to each well and the colorimetric reaction was allowed to proceed for 2 min. A 1 M phosphoric acid solution (100 μl) was added to each well to quench the reaction and the plate was placed into an ELISA plate reader. Absorbance was read at 450 nm.

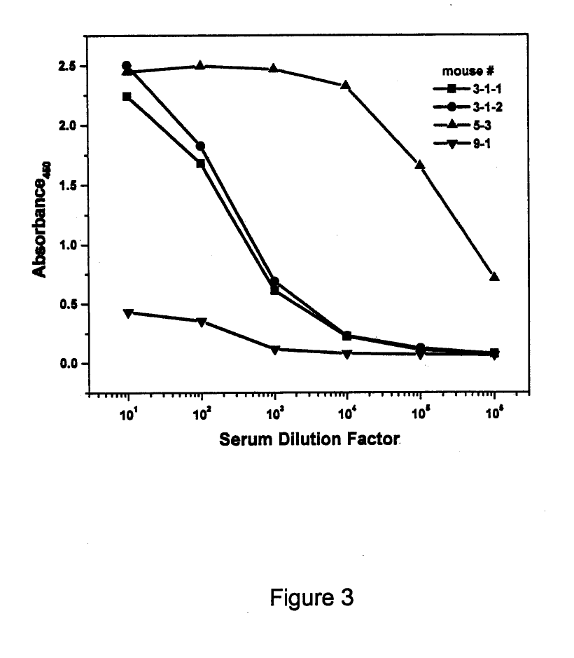

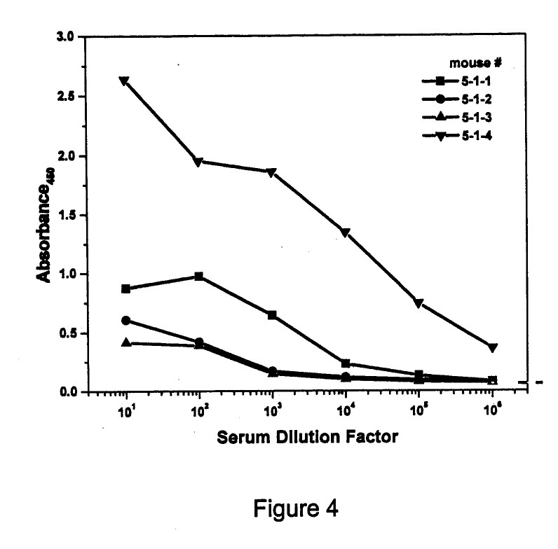

As shown in FIGS. 3 and 4, vaccinated mice exhibited strong IgG specific responses against the trisaccharide and tetrasaccharide conjugate vaccines. Although some mice responded with titers as high as those observed in rabbits, the titers were on average lower (i.e., 1:5,000-10,000 for mice and 1:50,000-100,000 for rabbits).

### Example 33

### Immunization of Experimental Rabbits with Trisaccharide-Tetanus Toxoid and Tetrasaccharide-Tetanus Toxoid Conjugates 26 and 27 and Titers by ELISA

Groups of New Zealand white rabbits were immunized with a tetanus toxoid conjugates 26 and 27 mixed with alum, as described above, on day 0 and day 21. A total of 0.3 mg of conjugate in 1 ml alum suspension was given on each day, distributed as follows: 200 μl at 2 intramuscular sites selected from the quadriceps/posterior thigh and lumbar muscles and 200 μL at 3 subcutaneous sites (scruff, flank). Blood was collected on day 28.

For antibody titration, a 96-well Nunc-Immuno ELISA plate (MaxiSorp F96) was coated with 100 μl of the BSA oligosaccharide conjugates (5 μg/mL in PBS buffer) and allowed to sit at 4C overnight. Excess solution was discarded, and the plate was washed 4 times with PBST. Serial ten-fold dilutions of immune sera starting at 1:10 and ending at 1:10⁶ were made in PBST containing 0.1% BSA and assayed together with pre-immune sera diluted over same range. The plate was incubated for 2 hours at room temperature and then washed 4 times with PBST. Bound antibody was detected using goat anti-rabbit IgG (H+L) conjugated to horse radish peroxidase (Kirkegaard and Perry Lab) at a working dilution of 1:2000 for 1 hour at room temperature. The plate was washed 4 times with PBST. A TMB solution (100 μl) was added to each well and the colorimetric reaction was allowed to proceed for 15 min. A 1 M phosphoric acid solution (100 μl) was added to each well to quench the reaction and the plate was placed into an ELISA plate reader and absorbance was read at 450 nm.

Previous work showed that when 10-50 μg of trisaccharide and tetrasaccharide conjugate vaccines were administered with Freunds adjuvant to New Zealand white rabbits on days 0, 21 and 28, exceptionally high titer of at least 1:500,000 were recorded against crude β-mannan coated ELISA plates.

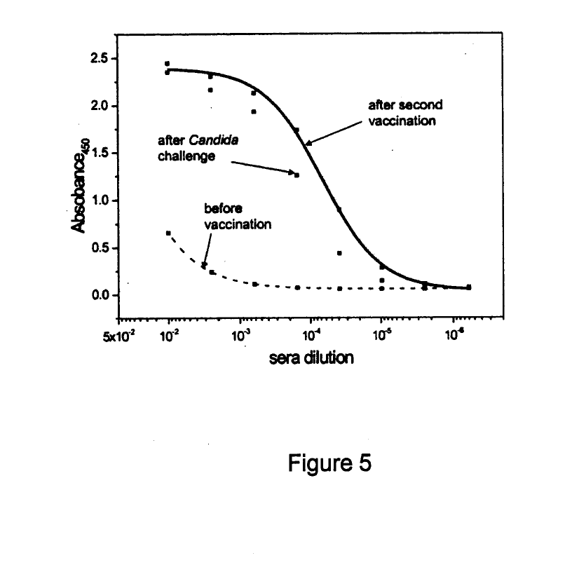

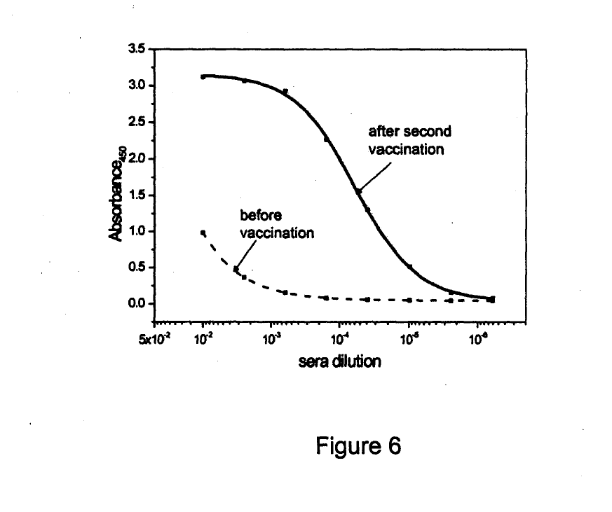

As shown in FIGS. 5 and 6, immunization with glycoconjugates presented with alum, an adjuvant approved for use in humans (Michon et al. (1998) Multivalent pneumococcal capsular polysaccharide conjugate vaccines employing genetically detoxified pneumolysin as a carrier protein. Vaccine 16:1732-1741) gave ELISA titers that consistently fell within the range 1:50,000-1:100,000 when measured against the homologous ligand attached to a heterologous protein BSA. A third injection did not improve titers.

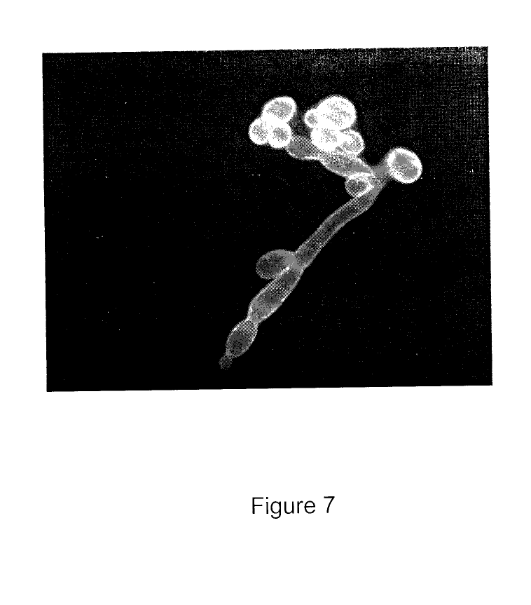

As shown in FIG. 7, antibody produced in response to the trisaccharide-tetanus toxoid vaccine strong stained Candida cells. At dilutions of 1:1000 and 1:10,000 pre-immune sera were negative while immune sera consistently stained both the hypha and budding cells of Candida albicans. Anti-sera to trisaccharide conjugates were just as effective in staining cells as the antisera raised against larger tetrasaccharide and hexasacharide antigen conjugates.

### Example 34

### Procedure for Establishing Central Venous Catheter Access in Rabbits for the Study of Invasive Candida Infections

Investigation of parameters surrounding invasive Candida infections and treatment with antifungal agents in animal models have most commonly used rabbits, which are large enough to be handled easily, and have sufficiently large veins to allow long-term central venous access. Central venous catheters are introduced to provide access for administration of immunosuppressive agents, antibacterial and antifungal agents, and to allow withdrawal of blood samples. We have used a less invasive modification of this approach involving catheterization of the marginal ear vein (Martin et al. (1991) A method for catheterizing rabbit vena cava via marginal ear vein. Lab. Anim. Sci. 41:493-494).

In this method, the animal was restrained without anesthetic in a simple restraining bag. The dorsal surface of the ear near the marginal ear vein was shaved, the ear surface was washed with hibitane, and 70% isopropanol was applied at the site of catheter insertion. The ear was then warmed using a 100-watt bulb for 10 minutes at a distance of approximately 20 cm. A 19 gauge needle was inserted into the marginal ear vein at a point suitable for positioning of the catheter (about half way down the ear). The needle was then removed and a 22 gauge PICC catheter (Becton Dickinson, Baltimore, Md.) was inserted into the opening in the ear vein. The catheter was advanced along the vein slowly until the hub of the catheter is at the point of entry (approximately 18 cm).

The catheter guide wire was then removed and the line was flushed with about 0.1 ml of heparinized saline. The placement of the catheter was tested by aspirating 1 ml of blood from the line. A PRN adapter was attached to the catheter hub and the septum was filled with about 0.2 ml of heparinized saline. The catheter hub was then secured to the ear by making a small butterfly with tape around the catheter hub. The tape was secured with a single stitch to the ear. The catheter was then taped to the ear and a small collar was placed around the animal's neck to prevent it from scratching the ear. Streptokinase was administered as required to prevent blockage in the lumen of the catheter.

For injection and sampling from the marginal ear vein, the PRN adapter was exposed and the tip of the adapter was wiped with alcohol. 0.2 ml of heparinized saline was flushed through the adapter. For collection of samples, the adapter was removed and a 22 gauge needle and syringe are used to aspirate blood. The PRN adapter was then returned and the line was again flushed with 0.2 ml of heparinized saline. The same procedure was followed for administration of organisms, immunosuppressive agents, and antibacterial or antifungal agents.

Immunosuppression in rabbits was achieved by intravenous administration of cyclophosphamide (200 mg/kg/day on days 28, 32 and 36 (induction), and daily doses of triamcinolone acetonide (10 mg/day) subcutaneously from day 28 though day 42 (maintenance). Additional triamcinolone can be given to extend the maintenance period. With this schedule granulocytopenia can be continued for the full course of the experimental period.

Total white blood cell counts were determined by particle counting (Coulter Electronics, Mississauga, ON) and the percentage of granulocytes and determination of granulocytopenia was measured by differential counts in peripheral blood smears.

To prevent bacterial infections, ceftazidime (150 mg/kg/day iv; GlaxoSmithKline, Collegeville, Pa.) and/or vancomycin (15 mg/kg/day iv; Eli Lilly, Indianapolis, Ind.) were administered starting at day 4 of chemotherapy and continued throughout the course of granulocytopenia.

### Example 35

### Candida Challenge Experiments

Investigation of the parameters surrounding invasive Candida infections and treatment with antifungal agents in animal models most commonly use a rabbit model (Walsh et al. (1988) Chronic silastic central venous catheterization for induction, maintenance and support of persistent granulocytopenia in rabbits. Lab Anim. Sci. 38:467-471; Walsh et al. (1992) Experimental antifungal chemotherapy in granulocytopenic animal models of disseminated candidiasis: approaches to understanding investigational antifungal compounds for patients with neoplastic diseases. Clin Infect Dis. 14 Suppl 1: S 139-S147). The neutropenic rabbit model, which simulates the immunocompromised patient, was used in the following experiment.

Groups of 6 New Zealand white rabbits were immunized with tetanus toxoid conjugates 26 mixed with alum, as described above, on days 0 and 21. Alternatively, groups of rabbits were given tetanus toxoid as a control, administered under identical conditions. A total of 0.3 mg of conjugate (or toxoid in the case of controls) in 1 ml alum suspension was given on days 0 and 21, distributed as follows: 200 μL at 2 intramuscular sites (quadriceps/posterior thigh, lumbar muscles and 200 μL at 3 subcutaneous sites (scruff, flank). Blood was collected on day 28.

Rabbits were catheterized via the marginal ear vein and immunosuppression was established according to the above-described protocols. On occasion, catheters were inserted between days 30 and 35, in which case the immunosuppression protocol was shifted by the appropriate number of days.

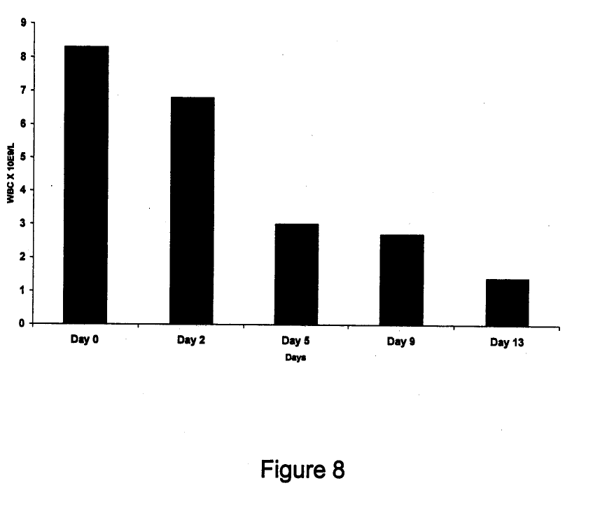

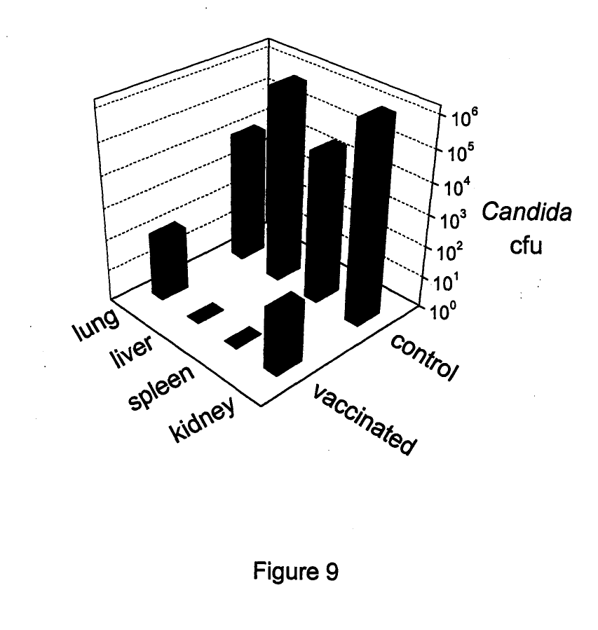

At day 28 cyclophosphamide was given to induce neutropenia (a decrease in white cells; as shown in FIG. 8). After 6 days, the antibody titer was reduced but did not drop more than 50% of its value on day 28 (FIG. 5). Antiobiotics were given to prevent bacterial infection, as required. At day 34, the rabbits were challenged by an intravenous infusion of 10³ cfu of live Candida albicans. At about day 40 (i.e., about 5-7 days following Candida infection), or when the condition of the rabbits so warranted, the experiment was terminated and a Candida count was determined for the organs; spleen, liver, kidney and lungs. The protection data shown in FIG. 9 represents the results of 5 rabbits having received glycoconjugate vaccine and 3 control rabbits immunized with tetanus toxoid.

### Example 36

### Additional Candida Challenge Experiments

### Immunizations of Rabbits

White New Zealand female rabbits (weighing approximately 3 kg ) were immunized twice, at day 0 and day 21 days, with a trisaccharide-TT conjugate with alum −0.3 mg of conjugate in 1 ml of alum suspension in PBS per rabbit. Injections of 0.2 ml were made into quadriceps/posterior thigh, lumbar muscles (both sides) and 3 subcutaneous sites. Control animals were injected with an identical formulation of tetanus toxoid alone. On day 28, 7 days after the second immunization blood samples were collected and analyzed for IgG titer by ELISA.

### Catheterization

Rabbits were catheterized (9-14 days after the second vaccination) through the marginal ear vein using a fluoroscopic pediatric intracatheter. The catheter was threaded carefully through the venous system until it reached the right atrium (approximately 28 cm). With a PRN heparin/saline lock adapter attached to the catheter hub, and a small Elizabethan collar in place, the procedure was complete. Daily heparin flushes were given to prevent catheter clogging.

### Protection Experiment

After one day of rest, animals were immunocompromised by administration of 200 mg of cyclophosphamide and maintained with a daily dose of triamcinolone—10 mg S.C.

Cyclophosphamide was administered again on the day 4 and 8 after catheterization. Antibiotics; vancomycin (15 mg/kg IV) and ceftazidime (150 mg/kg IV) were administered daily, starting from day 4 after catheterization. To induce disseminated candidiasis, animals were challenged with live Candida (1×10³ cells in 100 μl of sterile PBS) on the day 6 after catheterization. After 14 days from catheterization rabbits were euthanized and tissue samples of kidney, liver, spleen and lungs were taken for analysis for presence of live Candida albicans cells. See Table 5.

TABLE 5
Flow chart for immunosuppression and Candida challenge experiment

| Day | Catheter in | Hep Flush | Cyclophosphamide | Triamcinolone | Candida | Antibiotics | Blood Sample |
|---|---|---|---|---|---|---|---|
| 0 | X | X | | | | | X |
| 1 | | X | X | X | | | |
| 2 | | X | | X | | X | |
| 3 | | X | | X | | X | |
| 4 | | X | X | X | | X | |
| 5 | | X | | X | | X | |
| 6 | | X | | X | X | X | X |
| 7 | | X | | X | | X | |
| 8 | | X | X | X | | X | |
| 9 | | X | | X | | X | X |
| 10 | | X | | X | | X | |
| 11 | | X | | X | | X | |
| 12 | | X | | X | | X | |
| 13 | | X | | X | | X | |
| 14 | | | | | | | X |
| Necropsy | | | | | | | |
| Day | | | | | | | |

### Tissue Sample Analysis for Candida Counts

Samples of kidney, liver, spleen and lungs were weighted in empty Kendall Precision disposable tissue grinder container and homogenized with 0.5 ml of BHI broth. Homogenate was then transferred to 4.5 ml of BHI and vortexed. Empty container was weighed again to obtain exact weight of the tissue sample. The sample was then serially diluted up to 10E-7 (10× each time) by transferring 0.5 ml of the sample to 4.5 ml of BHI broth. 100 μl from each dilution was plated in duplicates on Sheep Blood Agar plates.

Colonies were counted after 20-24 hours of incubation on plates showing 50-100 colonies. CFU was calculated according to formula:

### Average Colony×Reciprocal of Dilution ×10×4.5 Weight of Tissue in Grams

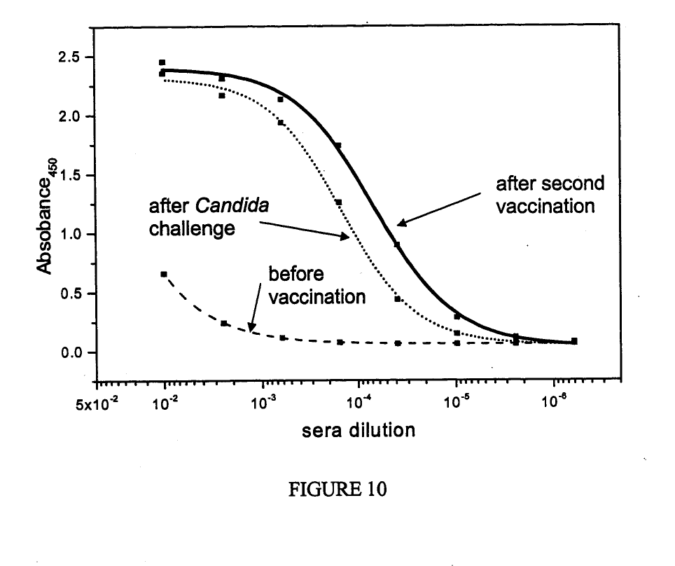

FIG. 10 shows comparison of viable C. albicans cells counts in different organs, 8 days after challenge with live fungi. Given values are the number of cfu (colony forming units) per gram of tissue. “Vaccinated” bars represent average values for rabbits vaccinated with trisaccharide conjugate. “Control” bars refer to a control group, vaccinated with tetanus toxoid.

Statistical analysis conducted with the Generalized Estimating Equation showed significant reduction of Candida counts in the kidney and liver with no statistically significant effect for spleen and lungs. The statistical analysis is summarized below:
Control/Vaccinated ratio of Candida in examined organs:
Kidney: 9.3, p=0.016; Liver: 193.6, p=0.035; Spleen: 1.3, p=0.8; Lungs: 20.4, p=0.99. (p-statistical significance value)

### Preparation of Candida Inoculum

Candida albicans ATCC strain 3153A was subcultured one day prior inoculum preparation on SDA medium (Saboraud Dextrose Agar). Fresh culture (18-24 h) was used for preparation of 0.5 McFarland suspension in sterile saline (0.5 McFarland standard suspension=1.5×10E6 CFU/ml) using Vitek colorimeter. The suspension diluted in PBS to required CFU was used for rabbits inoculation.

### ELISA Protocol

Polystyrene 96 wells plates were coated overnight with trisaccharide-BSA conjugate at concentration 5 μg/ml in PBS. After washing with PBS containing 0.1% Tween (PBST) wells were filed with 100 μL of serial dilutions of sera (starting from 10E-3) in root often order. BSA (0.1%) in PBST was used for dilutions to prevent unspecific binding. Plate were sealed and incubated for 2 h at room temperature. After washing with PBST, a reporter antibody (anti-Rabbit IgG, HRP conjugate, KPL) in 0.1% BSA PBST, at dilution 1/2000 was applied and plates were incubated for 1 h at room temperature. Plates were washed again with PBST and color developed with HRP substrate system (KPL) for 15 min. Reaction was stopped with 1M phosphoric acid and absorbance measured in ELISA reader.

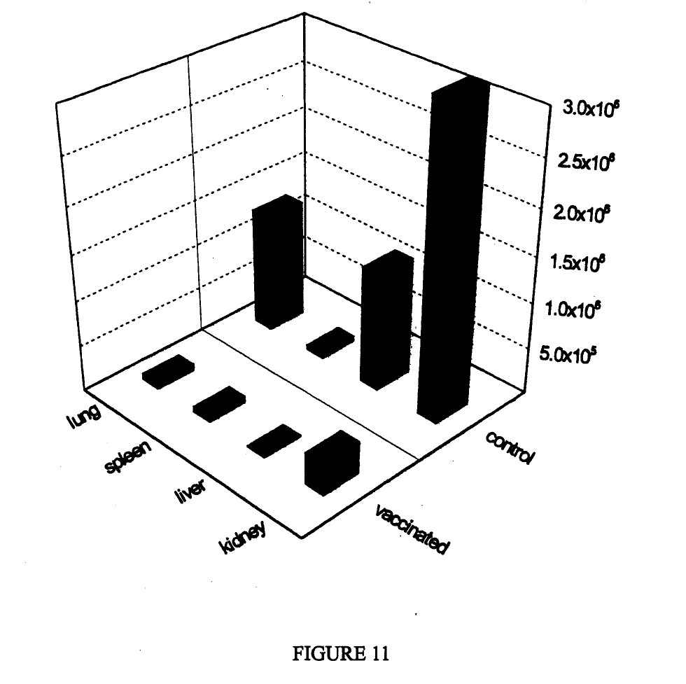

FIG. 11 shows a graph of relative antibodies in rabbits immunized with trisaccharide-BSA conjugate. Here, rabbits were vaccinated twice with tetanus toxoid glycoconjugate absorbed on alum, an adjuvant approved for use in humans. Titers were assayed against trisaccharide-BSA conjugate. Control sera from animals injected with tetanus toxoid did not show specific anti-mannan activity and are not plotted for clarity.

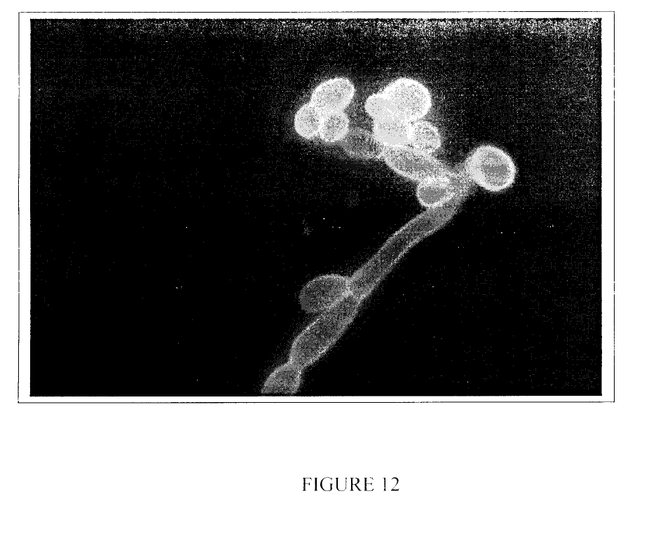

FIG. 12 shows immunofluorescent staining of C. albicans cells using rabbit antiserum raised against trisaccharide tetanus toxoid conjugate. Antibodies bind to antigen presented on the walls of Candida hypae and budding cells.

One skilled in the art will recognize that the above Examples are for exemplification only and should in no way limit the scope of the invention. Modifications of the compositions and methods described or referred to, herein, will be apparent to one skilled in the art, without departing from the scope of the invention.

### REFERENCES

The following publications, patents and patent applications are cited in this application as superscript numbers. Moreover, all of the above publications, patents and patent applications are herein incorporated by reference in their entirety to the same extent as if each individual publication, patent or patent application was specifically and individually indicated to be incorporated by reference in its entirety.
1. Casadevall, A.; Cassone, A.; Bistoni, F.; Cutler, J. E.; Magliani, W.; Murphy, J. W.; Polonelli, L.; Romani, L. (1998) Med. Mycol. 36, Suppl. 1, 95-105.
2. Beck-Sague, C. M.; Jarvis, W. R., (1993) J. Infect. Dis. 167, 1247-1251.
3. Landman, D.; Saurina, G.; Quale, J. M., (1998) Clin. Infect. Dis. 26:183-184.
4. Levitz, S. M., (1992) Clin. Infect. Dis., 14 (Suppl. 1), S37-42.
5. Jouault, T.; Delaunoy, C.; Sendid, B.; Ajana, F.; and Poulain, D., (1997) Clin. Diag. Lab. Immun. 4, 328-333.
6. Shibata, N., Kobayashi, H., Tojo, M., & Suzuki, S. (1986) Arch.

Biochem. Biophys. 251, 697-708.
7. Shibata, N.; Fukasawa, S.; Kobayashi, H.; Tojo, M.; Yonezu, T.; Ambo, A.; Ohkubo, Y.; Suzuki, S., (1989) Carbohydr. Res. 187, 239-253.
8. Han, Y.; Cutler, J. E., (1995) Infect. Immun. 63, 2714-2719.
9. Han, Y.; Cutler, J. E., (1997) J. Infect. Dis. 175, 1169-1175.
10. Han, Y., Riesselman, M. H.; Cutler, J. E., (2000) Infect. Immun. 68, 1649-1654.
11. Han, Y.; Kozel, T. R.; Zhang, M. X.; MacGill, R. S.; Carroll M. C.; Cutler. J. E., (2001) J. Immunol. 167, 1550-1557.
12. Han, Y.; Kanbe, T.; Cherniak, R.; Cutler, J. E., (1997) Infect. Immun., 65, 4100-4107.
13. Trinel, P-A.; Plancke, Y.; Gerold, P.; Jouault, T.; Delplace, F.; Shwarz, R. T.; Strecker, G.; Poulain, D., (1999) J. Biol. Chem. 274, 30520-30526.
14. Trinel, P. A.; Lepage, G.; Joulault, T.; Strecker, C.; Poulain, D., (1997) FEBS Lett., 416, 203-206.
15. Han, Y.; Ulrich, M. A.; Cutler, J. E., (1999) J. Infect. Dis. 179, 1477-1484.
16. Gridley, J. J.; Osbom H. M. I., J. Chem. Soc. Perkin Trans 1, 2000, 1471-1491.
17. Barresi, F.; Hindsgual, O. Synthesis of β-D-mannose Containing Oligosaccharides. In Modem Methods in Carbohydrate Synthesis, Khan, S. H.; O'Neil, R. A. Eds; Harwood Academic Publishers: Amsterdam, 1996, 251-276.
18. Lergenmüller, M.; Nukada, T.; Kuramochi, K.; Akhito, D.; Ogawa, T.; Ito, Y.; 30 Eur. J. Org Chemz. 1999, 1367-1376.
19. (a) Crich, D.; Sun, S. J. Am. Chem. Soc. 1998, 120, 435. (b) Crich, D.; Li, H.; Yao, Q.; Wink, D. J.; Sommer, R. D.; Rheingold, A. L. J. Am. Chem. Soc. 2001, 123, 5826.
20. Nitz, M.; Purse, B. W.; Bundle, D. R., (2000) Org. Lett., 2, 2939-2943.
21. Bundle, D. R.; Nitz, M., (2001) J. Org. Chem., 66, 8411-8423.
22. Nitz, M.; Ling, C.-C.; Otter, A.; Cutler, J. E.; Bundle, D. R., (2002) J. Biol. Chem., 277(5), 3440-3446.
23. Nitz, M. 2001. Ph.D. thesis. Synthesis of Trichinella spiralis and Candida albicans antigens, containing β-mannopyranosyl epitopes, their conformation and immunochemistry. University of Alberta, Edmonton, Alberta, May 4.
24. Nitz, M.; Bundle, D. R. J. Org. Chem. 2001, 66, 8411.
25. Charette, Andre B.; Turcotte, N.; Cote, B. J. Carbohydr. Chem. 1994, 13, 421.
26. Schmidt, R. R.; Effenberger, G. Liebigs Ann. Chem. 1987, 825.
27. Grathwohl, M.; Schmidt, R. R. Synthesis 2001, 2263.
28. Wu, X.; Ling, C. C.; Bundle, D. R. Org. Lett. 2004, 6, 4407.
29. Rodebaugh, R.; Debenham, J. S.; Fraser-Reid, Snyder, J. P.; J. Org. Chem., 1999, 64, 1758-1761.
30. Kitov, P. I., Tsvetkov, Yu. E, Bakinovsky, L. V. Dokl. Chem. (Eng. Trans.) 1993, 329, 1-3.
31. U.S. Pat. No. 5,969,130.
32. Martin F. R. et al. (1991) Lab. Anim. Sci. 41:493-494).
33. Michon, F. et al. (1998) Vaccine 16:1732-1741.
34. Walsh, T. J. et al. (1988) Lab Anim. Sci. 38:467-471.
35. Walsh, T. J. et al. (1992) Clin Infect Dis. 14 Suppl 1: S139-S147.
36. Remington's Pharmaceutical Sciences, Mace Publishing Company, Philadelphia Pa. 17.sup.th ed. (1985)
37. The National Formulary XIV (1975) 14^{th} Ed., American Pharmaceutical Association, Washington, DC (1975).
38. The Pharmacological Basis for Therapeutics (7th ed.).
39. Shibata, N.; Arai, M.; Haga, E.; Kikuchi, T.; Najima, M.; Satoh, T.; Kobayashi, H.; Suzuki, S. Infect. Immun. 1992, 60, 4100.
40. Shibata, N.; Kobayashi, H.; Okawa, Y.; Suzuki, S. Eur. J. Biochem. 2003, 277, 3400.
41. (a) Wu, X.; Grathwohl, M.; Schmidt, R. R. Angew. Chem. Int. Ed. 2002, 41, 4489. (b) Wu, X.; Schmidt, R. R. J. Org. Chem. 2004, 69, 1853.
42. (a) Hindsgaul, O.; Norberg, T.; Le Pendu, J.; Leimieux, R. U. Carbohydr. Res. 1982, 109, 109. (b) Pozsgay, V.; Chu, C.; Pannell, L.; Wolfe, J.;

Robbins, J. B.; Schneer, R. Proc. Natl. Acad. Sci. U. S. A. 1999, 96, 5194.
43. (a) Kamath, P.; Diedrich, P.; Hindsgaul, O. Glycoconjugate J. 1996, 13, 315. (b) Andersen, S. M.; Ling, C. C.; Zhang, P.; Townson, K.; Willison, H. J.; Bundle, D. R. Org. Biomol. Chem. 2004, 2, 1199.
44. (a) Mawas, F.; Niggemann, J.; Jones, C.; Corbel, M. J.; Kamerling, J. P.; Vliegenthart, Johannes F. G. Infect. Immun. 2002, 70, 5107. (b) Buskas, T.; Li, Y.; Boons, G.-J. Chem. Eur. J. 2004, 10. 3517.

1. A conjugate comprising:
a plurality of oligosaccharides comprising a (1→2)-β-D-mannopyranose or a (1→2)-β-D-mannopyranose derivative wherein each monosaccharide unit of said oligosaccharide is linked via an inter-glycosidic atom by an oxygen or a sulfur;
a protein carrier; and
a linking group derived from a linking agent;
wherein said linker group covalently attaches each of said plurality of oligosaccharides to said protein carrier.

2. A conjugate of claim 1, wherein the linking agent has at least three sites of attachment, one of which is for covalent attachment to the protein carrier.

3. A conjugate of claim 2, wherein at least two sites of attachment comprise hydroxyl groups.

4. A conjugate of claim 2, wherein the linking agent comprises one to twenty atoms at its longest chain.

5. A conjugate of claim 2, wherein the linking agent comprises a functional group selected from the group consisting of hydroxyl, amine, sulfonyl halide, carboxyl, acyl azide, epoxide, maleimide, and carbonate.

6. A conjugate of claim 2, wherein the linking agent comprises a functional group selected from the group consisting of isocyanate, epoxide, ketone, amine, alkyl halide, aryl halide, alcohol, sulfhydryl, and aminooxy.

7. A conjugate of claim 1, wherein the linking agent is a dicarboxylic acid.

8. A conjugate of claim 7, wherein the linking agent is adipic acid or azelaic acid.

9. A conjugate of claim 8, wherein the linking agent is a p-nitrophenyl adipic acid diester.

10. A conjugate of claim 9, wherein the linking agent comprises a sugar having at least one free hydroxyl.

11. A conjugate of claim 10, wherein the linking group comprises a glucose.

12. The conjugate of claim 1 having the structure:

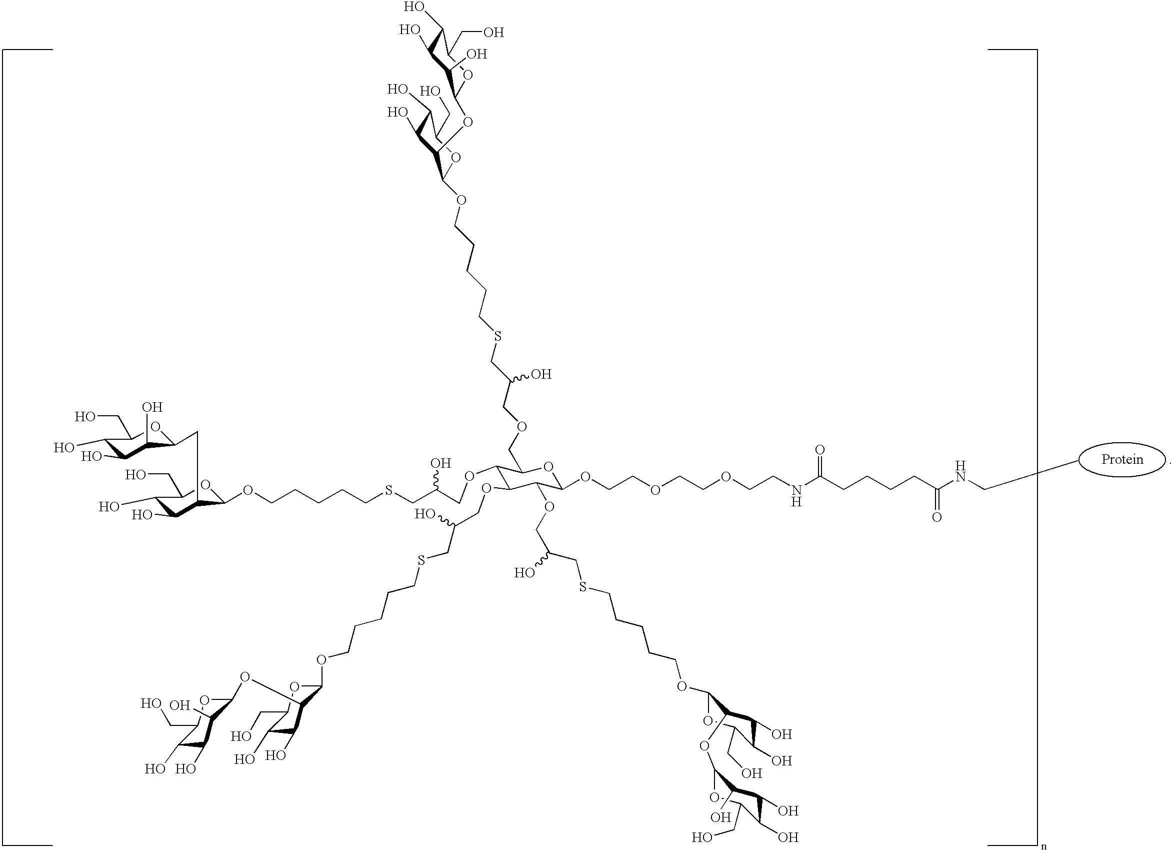

13. A conjugate of claim 1, wherein the oligosaccharide is selected from the group consisting of disaccharide through hexasaccharide of (1→2)-β-D-mannopyranose and disaccharide through hexasaccharide of (1→2)-β-D-mannopyranose derivatives.

14. A conjugate of claim 13, wherein the oligosaccharide is β-D-mannopyranose-(1→2)-β-D-mannopyanose-(1→2)-β-D-mannopyranose.

15. A conjugate of claim 13, wherein the oligosaccharide is β-D-mannopyranose-(1→2)-β-D-mannopyranose.

16. A conjugate of claim 1, wherein the protein carrier comprises at least one or more lysine side chains.

17. A conjugate of claim 1, wherein the protein carrier is selected from the group consisting of tetanus toxoid/toxin, diphtheria toxoid/toxin, bacteria outer membrane proteins, crystalline bacterial cell surface layers, serum albumin, gamma globulin, and keyhole limpet hemocyanin.

18. A conjugate of claim 1, wherein the protein carrier is selected from the group consisting of bovine serum albumin, human serum albumin, tetanus toxoid, a recombinant outer membrane class 3 porin (rPorB) from group B Neisseria meningitidis, and T-cell peptide carriers.

19. A conjugate of claim 1, wherein the Candida species is Candida albicans.

20. An immunogen comprising the conjugate of claim 1, and a pharmaceutically acceptable carrier.

21. An immunogen of claim 20, further comprising a pharmaceutically acceptable adjuvant.

22. The composition of claim 21, wherein the pharmaceutically acceptable adjuvant is selected from the group consisting of alum, aluminum phosphate, aluminum hydroxide, aluminum sulfate, stearyl tyrosine, Freund's adjuvant, and RIBI's adjuvant.

23. Use of the conjugate of claim 1 in the preparation of a medicament to induce an immune response to a Candida species in a subject in need thereof.

24. A method for inducing an immune response against a Candida species in a mammal in need thereof comprising administering to said mammal an immunogenic effective amount of a conjugate of claim 1.

25. A method of claim 24, wherein the Candida species is Candida albicans.

26. A method of claim 25, wherein the conjugate is administered directly to a urogenital tract.

27. A method for ameliorating or preventing an infection by a Candida species in a mammal in need thereof comprising administering to said mammal an immunogenic effective amount of a conjugate of claim 1.

28. A method of claim 27, wherein the Candida species is Candida albicans.

29. A method of claim 28, wherein the conjugate is administered directly to a urogenital tract.
